(19) United States
(12) Patent Application Publication
(10) Pub. No.: US 2004/0073219 A1
(43) Pub. Date: Apr. 15, 2004

(54) Insertion instrument

(76) Inventors: Jeffry B. Skiba, Oracle, AZ (US); Jeffrey P. Baldwin, Phoenix, AZ (US); Dennis Colleran, North Attleborough, MA (US); Stefan Gabriel, Mattapoisett, MA (US); Justin Dye, Mansfield, MA (US)

Correspondence Address: JOEL PETROW, Chief Patent Counsel, SMITH & NEPHEW NORTH AMERICA, 1450 Brooks Road, Memphis, TN, 38116, US

(21) Appl. No.: 10/303,076

(22) Filed: Nov. 25, 2002

Related U.S. Application Data
(63) Continuation-in-part of application No. 10/270,262, filed on Oct. 15, 2002, now abandoned.

(51) Int. Cl. A61B 17/56

(52) U.S. Cl. 606/72

(57) ABSTRACT

A method of deploying a fixation mechanism includes providing an insertion instrument and a fixation mechanism, creating an insertion tunnel within tissue of a patient, and positioning the insertion instrument with the fixation mechanism in the insertion tunnel. The insertion instrument includes a main member connected to an instrument handle and a secondary member connected to a pushing handle and positioned within the main member. The main member and the secondary member extend along a longitudinal axis. The fixation mechanism includes a first portion coupled to the main member and a second portion coupled to the secondary member. The method includes translating the pushing handle along the longitudinal axis relative to the instrument handle such that translating causes the secondary member to move the second portion relative to the first portion.

### CROSS REFERENCE TO RELATED APPLICATIONS

This application is a continuation-in-part of U.S. patent application Ser. No. 10/270,262, titled “SOFT TISSUE LIGAMENT TO BONE FIXATION DEVICE WITH INSERTER” and filed Oct. 15, 2002, which claims benefit to U.S. patent application Ser. No. 09/439,148, titled “SOFT TISSUE LIGAMENT TO BONE FIXATION DEVICE WITH INSERTER” and filed Nov. 12, 1999, which claims the benefit of U.S. Provisional Application No. 60/108,087, filed Nov. 12, 1998; and of U.S. patent application Ser. No. 10/046,290, titled “TISSUE ANCHOR INSERTION TOOL” and filed Jan. 16, 2002. These applications are herein incorporated by reference.

### TECHNICAL FIELD

This description relates to surgical reattachment of soft tissue/ligament to bone.

### BACKGROUND

Current methods of re-attachment of soft tissue to bone include bone tunnels, surgical staples, surgical tacks, interference screws, and bone anchors. If the desired result is solely approximation of the soft tissue back to the bony insertion site, the aforementioned devices can be used within certain limitations. There are, however, certain tendons and ligaments that present the surgeon with a very specific set of constraints. For example, grafting of a tendon into the site of an irreparably torn anterior cruciate ligament in the human knee imposes particular constraints.

Many arthroscopic suture anchor/inserter systems are currently marketed. Current products include pre-assembled, disposable devices and devices for use with screw in or push in anchors.

### SUMMARY

In one general aspect, a fixation device for attaching soft tissue to bone includes a fixation mechanism, a shaft, and a securing mechanism that slides along the shaft. The securing mechanism may include an internal one-way locking mechanism.

In another aspect, a device for attaching soft tissue to bone includes a shaft, a fixation mechanism attached to the shaft, a one-way track in which the shaft may be inserted, and a securing mechanism for holding the shaft within the one-way track by compressing the securing mechanism against the shaft. The securing mechanism may have at least one of a conical shape, a cylindrical shape, a cubic shape, or a complex shape capable of exerting an adequate radial force against the shaft and into a surrounding bone. The fixation device may include a fixation mechanism with an expansion leg, a shaft with a one-way track, and a securing mechanism. The expansion leg of the fixation mechanism may be single or multiple legs or may be toggles, legs, expansion arms, barbs, tines, or other mechanisms to prevent backward translation. The one-way track of the shaft may be a single length or may be an adjustable length member having multiple lengths. The securing mechanism holds the tissue and has an internal one-way lock that slides along the one-way track. Alternatively, the fixation mechanism may include an inner core that expands as a result of the insertion of a device that causes radial displacement, such as, for example, a wedge, a tapered plug, or a screw.

A fixation device may be made of any biocompatible metal, such as titanium or stainless steel, plastic, such as nylon or polyester, or bioabsorbable, such as PLLA. Any material suitable for use in the body can be used. The material must provide adequate resistance to creep, hold the load required, and be unaffected by cyclic loading.

The fixation device supports a simple technique for implanting a fixation mechanism. The fixation mechanism can be controllably positioned for successful deployment, in that parts of the fixation mechanism deploy relative to one another for successful implantation. Additionally, the implantation device protects against jostling, dislodgement, and reorientation of the fixation mechanism anchor during implantation, with the means of the protection being retractable. The instrumentation can guide the fixation mechanism to the repair site through a cannula and/or incisions in the skin, fat layer, and other soft tissue.

In one general aspect, deploying a fixation mechanism includes providing an insertion instrument and a fixation mechanism, creating an insertion tunnel within tissue of a patient, and positioning the insertion instrument with the fixation mechanism in the insertion tunnel. The insertion instrument includes a main member connected to an instrument handle and a secondary member connected to a pushing handle and positioned within the main member. The main member and the secondary member extend along a longitudinal axis. The fixation mechanism includes a first portion coupled to the main member and a second portion coupled to the secondary member. During use, the pushing handle is translated along the longitudinal axis relative to the instrument handle such that the translating causes the secondary member to move the second portion relative to the first portion.

Implementations may include one or more of the following features. For example, translating may cause the secondary member to translate the second portion along the longitudinal axis relative to the first portion. Translating may cause the distal ends of the first portion to expand along a direction perpendicular to the longitudinal direction to a size having a radius greater than a radius of the insertion tunnel. Translating also may cause the distal ends of the first portion to enter tissue adjacent to the insertion tunnel. Translating may cause the distal ends of the first portion to engage tissue external to the insertion tunnel.

As another example, translating may cause the secondary member to rotate the second portion relative to the first portion about an axis perpendicular to the longitudinal axis. Translating may cause a width of the second portion to expand after rotation relative to a width before rotation to a width greater than a radius of the insertion tunnel. Translating may cause the ends of the second portion to enter tissue adjacent to the insertion tunnel. Translating also may cause the ends of the second portion to engage tissue external the insertion tunnel.

In another general aspect, a system for deploying a fixation mechanism includes an insertion instrument and a fixation mechanism. The insertion instrument has a main member connected to an instrument handle and a secondary member connected to a pushing handle and positioned within the main member. The main member and the secondary member extend along a longitudinal axis. The fixation mechanism has a first portion coupled to the main member and surrounding a second portion coupled to the secondary member. The insertion instrument and the fixation mechanism are coupled such that translation of the pushing handle along the longitudinal axis relative to the instrument handle causes the secondary member to move the second portion relative to the first portion and to expand distal ends of the first portion along a direction perpendicular to the longitudinal axis.

Other features will be apparent from the description, the drawings, and the claims.

### DESCRIPTION OF DRAWINGS

FIG. 1 shows a representation of a human knee with an ACL graft held in place with a fixation device.

FIGS. 2A-2V show examples of fixation mechanisms used in the fixation device of FIG. 1.

FIGS. 3A-3D show views of components of the fixation mechanism shown in FIG. 2A.

FIGS. 4A-4D show views of the fixation mechanism shown in FIG. 2B.

FIG. 5 shows a perspective view of the fixation mechanism shown in FIG. 2E disposed on a shaft.

FIGS. 6A-6D show views of a securing mechanism used in the fixation device of FIG. 1.

FIG. 7 shows a side view of a shaft and the securing mechanism disposed together.

FIG. 8 shows a side view of a tissue anchor insertion tool.

FIG. 9A shows an exploded view of the insertion tool of FIG. 8.

FIG. 9B shows an enlarged view of section 9B of FIG. 9A.

FIG. 10A illustrates a distal end of a cover of the insertion tool of FIG. 8.

FIG. 10B shows a cross-sectional side view of the cover of FIG. 10A.

FIG. 11 shows a cross-sectional side view of a shaft of the insertion tool of FIG. 8.

FIG. 12A illustrates a thumb contact region of the insertion tool of FIG. 8.

FIG. 12B shows a cross-sectional side view of the thumb contact region of the insertion tool of FIG. 8 taken along line 12B-12B of FIG. 12A.

FIG. 13 shows a cross-sectional side view of a distal region of the insertion tool of FIG. 8.

FIG. 14 illustrates a tissue anchor for use with the insertion tool of FIG. 8.

FIGS. 15A-15C show side views of the insertion tool of FIG. 8 shown at various stages during deployment of the tissue anchor.

FIGS. 16A and 16B illustrate deployment of the tissue anchor in bone using the insertion tool of FIG. 8.

FIG. 17 shows a cross sectional view of an insertion instrument.

FIGS. 18 and 19 show cross sectional views of the insertion instrument of FIG. 17 in which a fixation mechanism is attached and deployed.

FIG. 20 is a procedure performed by a surgeon for deploying a fixation mechanism using an insertion instrument.

FIGS. 21A-21C show cross sectional views of the fixation mechanism of FIGS. 18 and 19 at stages during deployment.

FIG. 22 shows a cross sectional view of an insertion instrument.

FIGS. 23 and 24 show cross sectional views of the insertion instrument of FIG. 22 in which a fixation mechanism is attached and deployed.

FIGS. 25A-25C show cross sectional views of the fixation mechanism of FIGS. 23 and 24 at stages during deployment.

FIG. 26 shows a perspective view of an insertion instrument that is deployed.

FIG. 27 shows a cross sectional view of the deployed insertion instrument of FIG. 26.

FIG. 28 shows a side view of the deployed insertion instrument of FIG. 26 in which a graft has been attached.

Like reference symbols in the various drawings indicate like elements.

### DETAILED DESCRIPTION

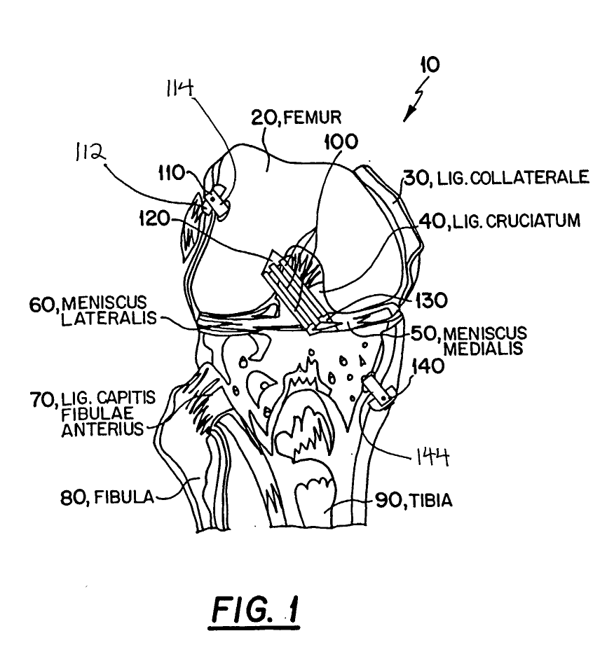

FIG. 1 is a representation of a human knee 10 with an ACL graft 100 (three lines) held in place in the joint space between the tibia 90 and the femur 20 by a fixation device. The fixation device includes two fixation mechanisms 110 and 140 and two securing mechanisms 120 and 130. Each of the fixation mechanisms 110 and 140 is attached to a respective shaft 114 or 144, which may be formed as a one-way locking strip. The shafts 114 and 144 extend through insertion tunnels from a corresponding fixation mechanism 110 or 140 to a corresponding securing mechanism 120 or 130. The graft 100 extends between and is held by the securing mechanisms 120 and 130.

For clarity, features of the human knee are labeled and briefly listed. FIG. 1 shows the right knee 10, from the front; the fibula 80; and interior ligaments, including the collateral ligament 30, the cruciate ligament 40, the medial meniscus 50, the lateral meniscus 60, and the anterior ligament 70 of the head of the fibula.

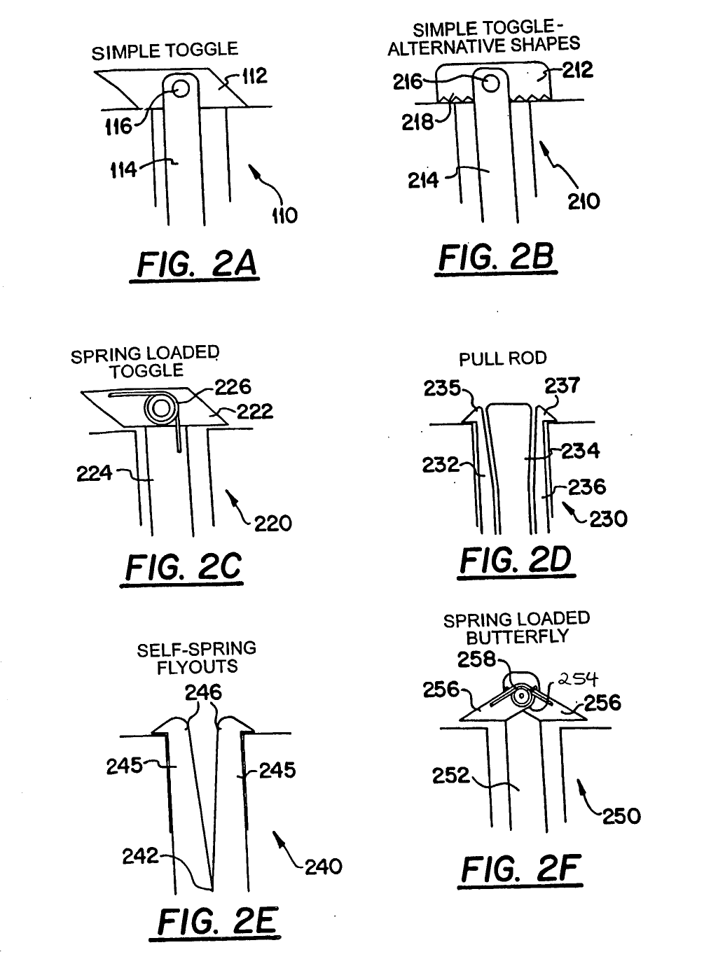

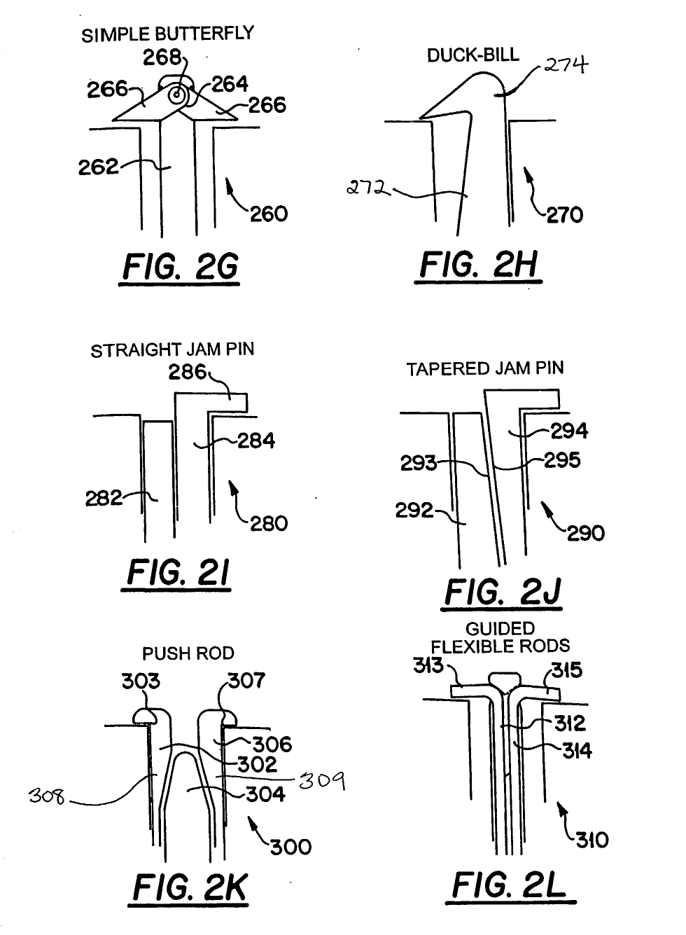

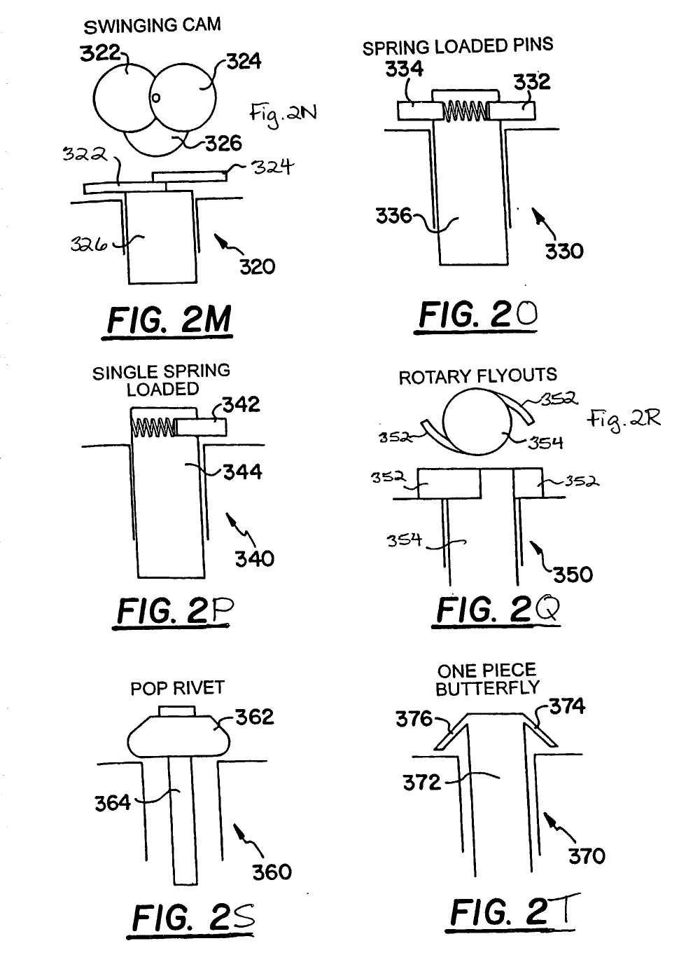

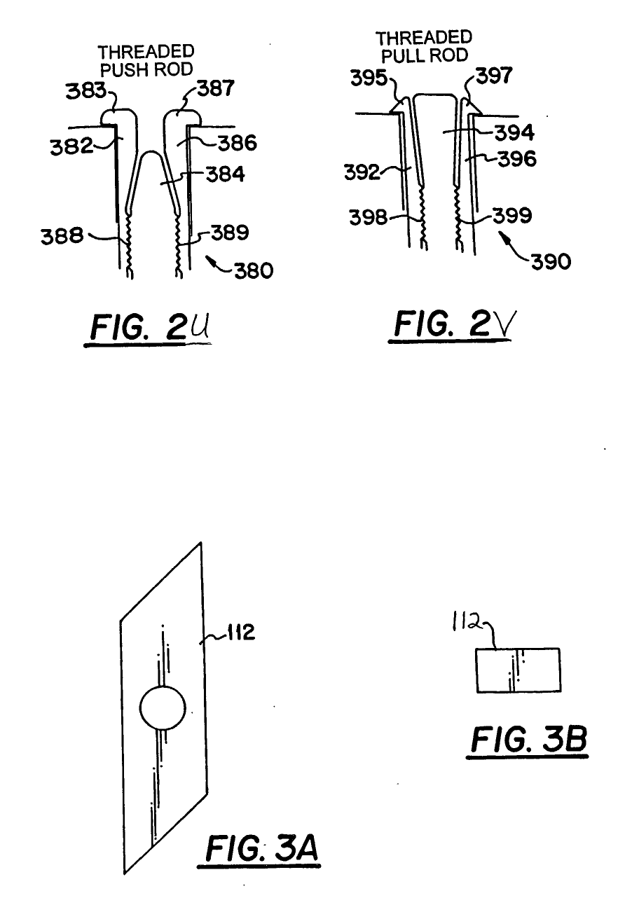

FIGS. 2A-2V illustrate examples of fixation mechanisms that may be used in the fixation device of FIG. 1. Each fixation mechanism provides resistance to the tensile forces exerted on the graft site. Generally, the fixation mechanism is designed to fit within the insertion tunnel and to expand upon actuation by the surgeon. For example, the fixation mechanism may slide through the insertion tunnel and then rotate so that a wider portion expands and grips an outer surface of the femur 20 (as shown in FIG. 1).

A fixation mechanism may include a toggle pin member or a toggle element, as shown, for example, in FIGS. 2A-2C. FIG. 2A illustrates a simple toggle fixation mechanism 110 having a fixation member 112 (see also FIG. 1) shaped as a parallelogram and attached to the longitudinal shaft 114. The longitudinal shaft 114, which is disposed in the insertion tunnel, may be attached to the fixation member 112 by a pin or screw 116 or the like.

FIG. 2B illustrates an alternative form of a simple toggle fixation mechanism 210 that has a rectangular fixation member 212 attached to a longitudinal shaft 214 by a pin or screw 216 or the like. The rectangular horizontal member 212 has teeth 218 disposed along one side.

FIG. 2C illustrates a spring-loaded toggle fixation mechanism 220 that has a fixation member 222 shaped as a parallelogram and attached to a longitudinal shaft 224 by a spring 226.

FIG. 2D illustrates a pull rod fixation mechanism 230 that includes three longitudinal shafts 232, 234, and 236. The longitudinal shafts 232 and 236 have duck-bill-like fixation portions 235 and 237 that extend over the edge of the insertion tunnel to hold the fixation mechanism in place. The longitudinal shafts 232 and 236 may be connected together at a proximal end (as shown in FIGS. 2E and 7).

FIG. 2E illustrates a self-spring flyout fixation mechanism 240 that includes two longitudinal members 245 connected together at their base 242. Each longitudinal member 245 has a duck-bill-like fixation portion 246 that extends over the edge of the insertion tunnel to hold the fixation mechanism in place.

FIG. 2F illustrates a spring-loaded butterfly fixation mechanism 250 that has a longitudinal shaft 252 and a butterfly-shaped fixation member 254 including two arms 256. The arms 256 of the butterfly-shaped fixation member 254 are connected together and to longitudinal shaft 252 by a spring 258.

FIG. 2G illustrates a simple butterfly fixation mechanism 260 that has a longitudinal shaft 262 and a butterfly-shaped fixation member 264 having two arms 266. The arms 266 of the butterfly-shaped fixation member 264 are connected together and to the longitudinal shaft 262 by a pin or screw 268 or the like.

FIG. 2H illustrates a duck-bill-shaped fixation mechanism 270. The fixation mechanism 270 includes a duck-bill shaped fixation member 274 connected to a shaft 272 such that the fixation member 274 extends over an edge of the insertion tunnel to hold the fixation mechanism 270 in place.

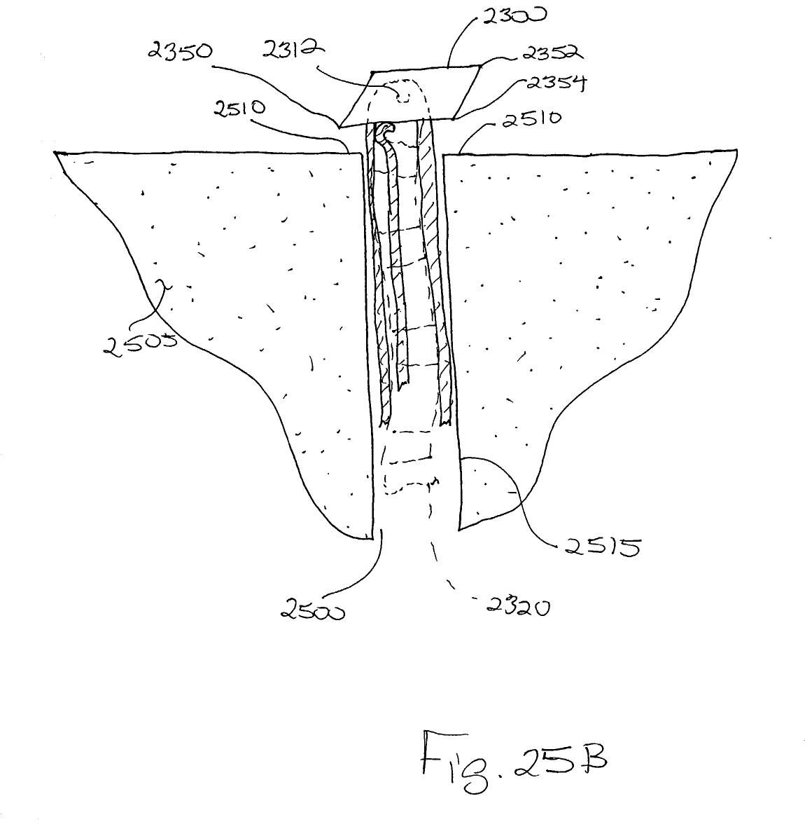

FIG. 21 illustrates a straight jam pin fixation mechanism 280 including a first longitudinal shaft 282 and a second longitudinal shaft 284 with a fixation member 286. The fixation member 286 extends over an edge of the insertion tunnel to hold the fixation mechanism in place.

FIG. 2J illustrates a tapered jam pin fixation mechanism 290 similar to the straight jam pin fixation mechanism 280 of FIG. 21. However, interior contacting sides 293 and 295 of the first longitudinal shaft 292 and the longitudinal portion of the second longitudinal shaft 294 are reciprocally tapered.

FIG. 2K illustrates a push rod fixation mechanism 300 that includes three longitudinal shafts 302, 304, and 306. The two outer longitudinal shafts 302 and 306 have respective fixation portions 303 and 307 that extend over the edges of the insertion tunnel to hold the fixation mechanism in place. The interior longitudinal shaft 304 has a blunt, tapered tip. The respective longitudinal portions 308 and 309 of the outer longitudinal shafts 302 and 306 are shaped around the blunt tip of the interior longitudinal shaft 304. The longitudinal shafts 302 and 306 may be connected together at a proximal end (as shown in FIGS. 2E and 7).

FIG. 2L illustrates a fixation mechanism 310 formed of guided flexible rods 312 and 314. The rods 312 and 314 have fixation portions 313 and 315 that extend over the edges of the insertion tunnel to hold the fixation mechanism in place. The flexible rods 312 and 314 may be connected together at a proximal end (as shown in FIGS. 2E and 7).

FIG. 2M illustrates a side view of a swinging cam fixation mechanism 320 and FIG. 2N illustrates a top view of the swinging cam fixation mechanism 320 of FIG. 2M. The fixation mechanism 320 includes three circular members 322, 324, and 326. The circular members 322 and 324 are disc-shaped and serve as fixation members while the circular member 326 is cylindrically-shaped. The circular members 322, 324, and 326 are concentrically arranged during insertion of the fixation mechanism 320 through the tunnel. As the circular members 322 and 324 clear the edge of the insertion tunnel, the circular members 322 and 324 swing out to form an eccentric arrangement and act to fix the mechanism in the insertion tunnel.

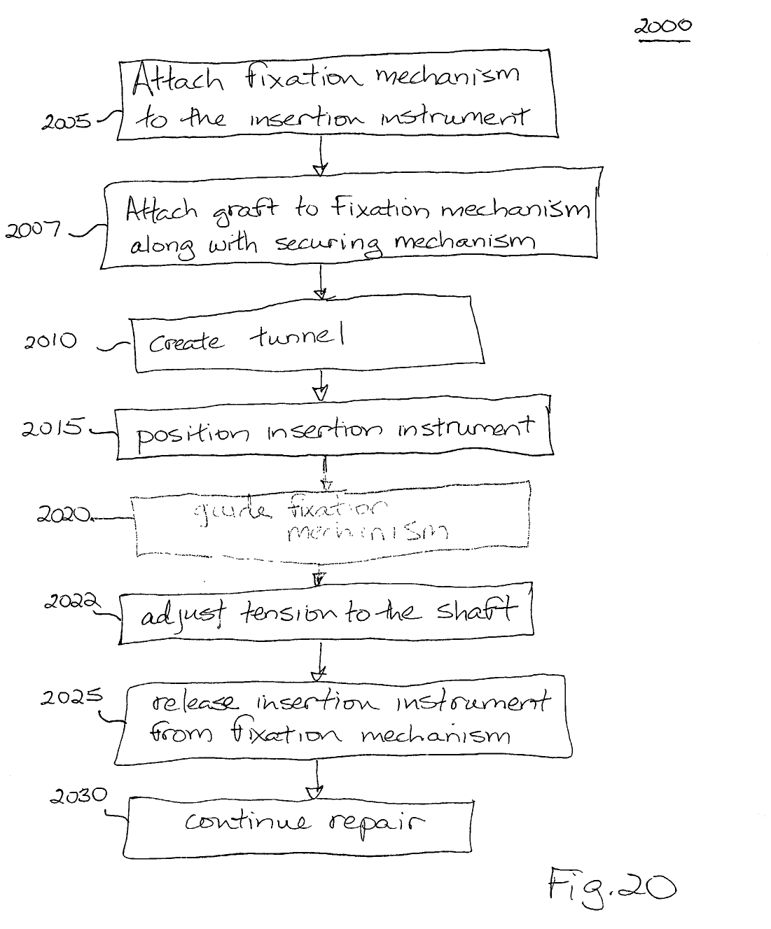

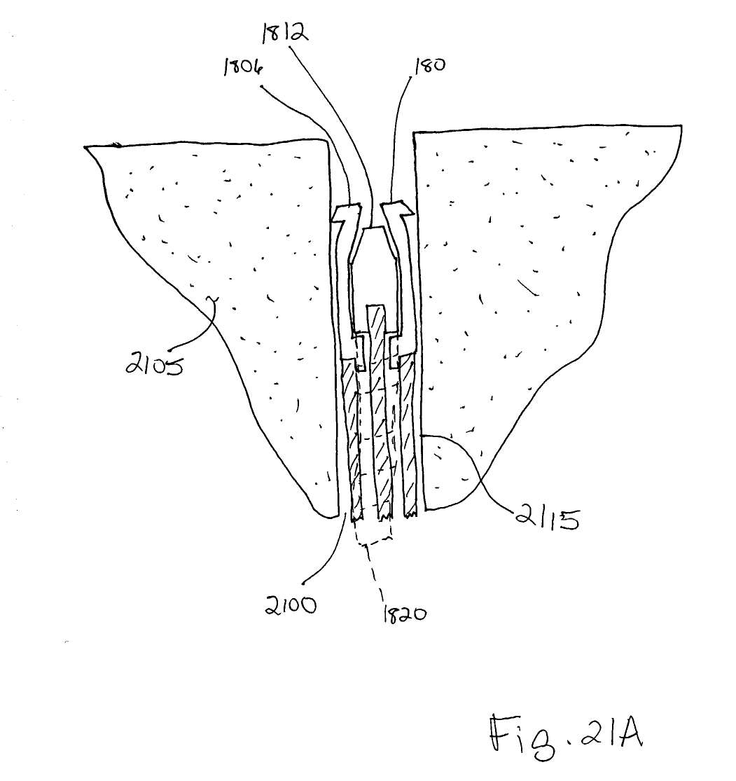

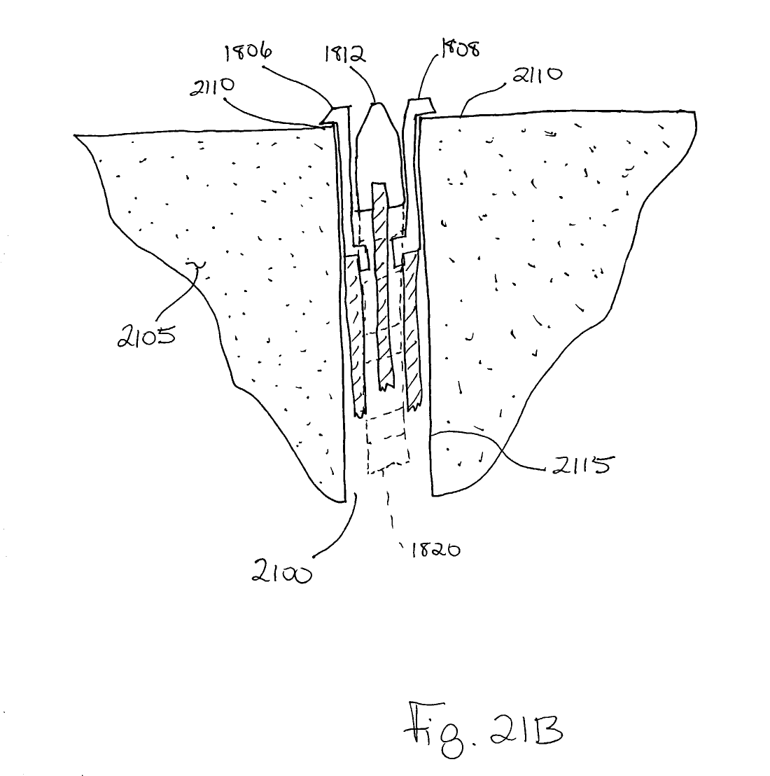

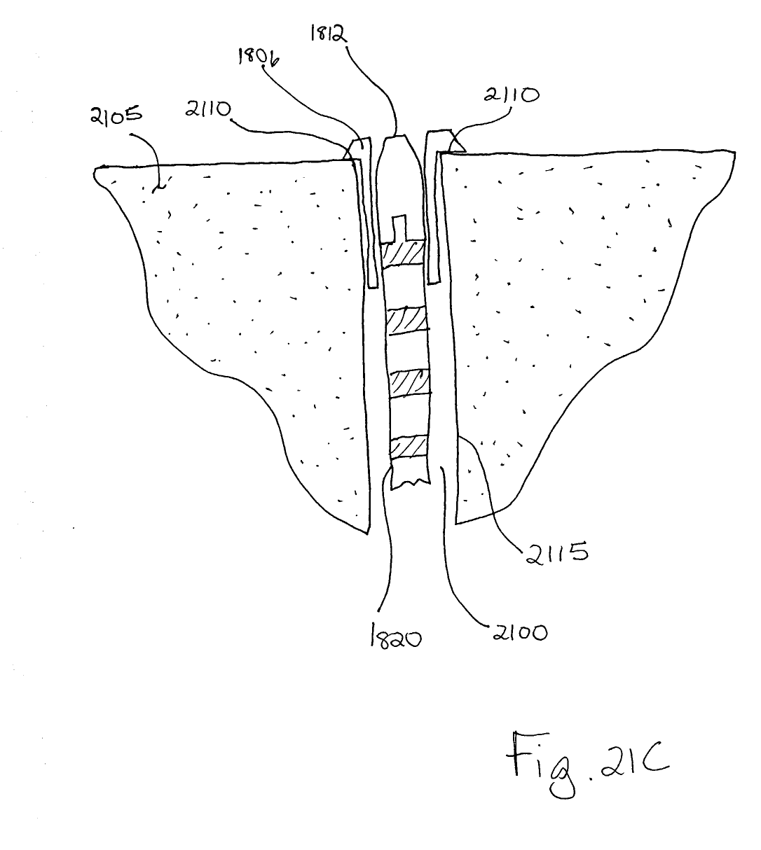

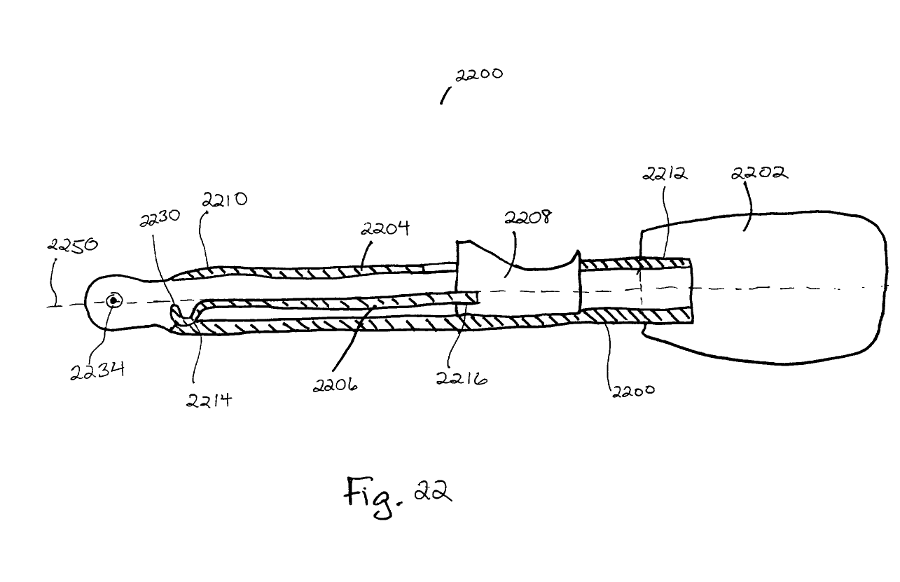

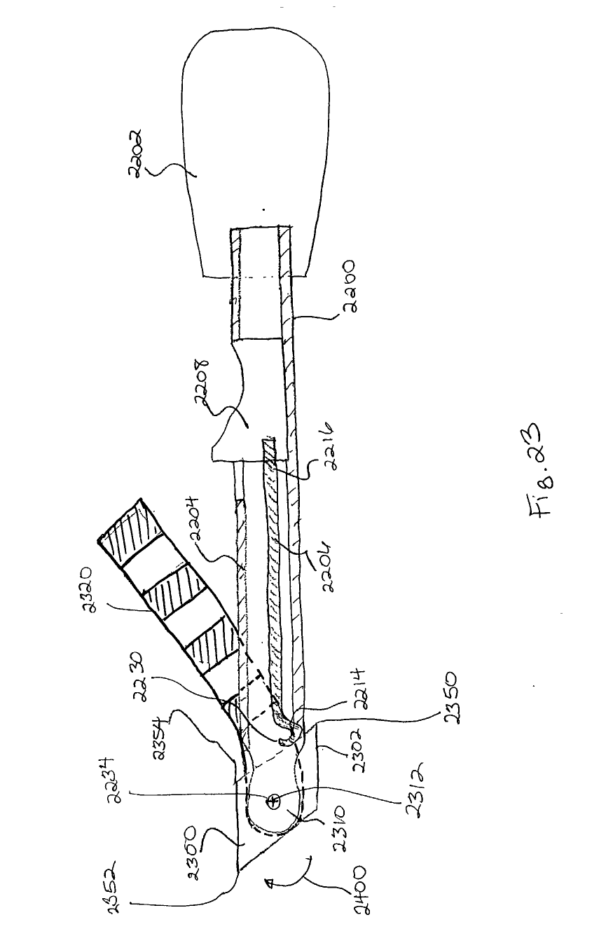

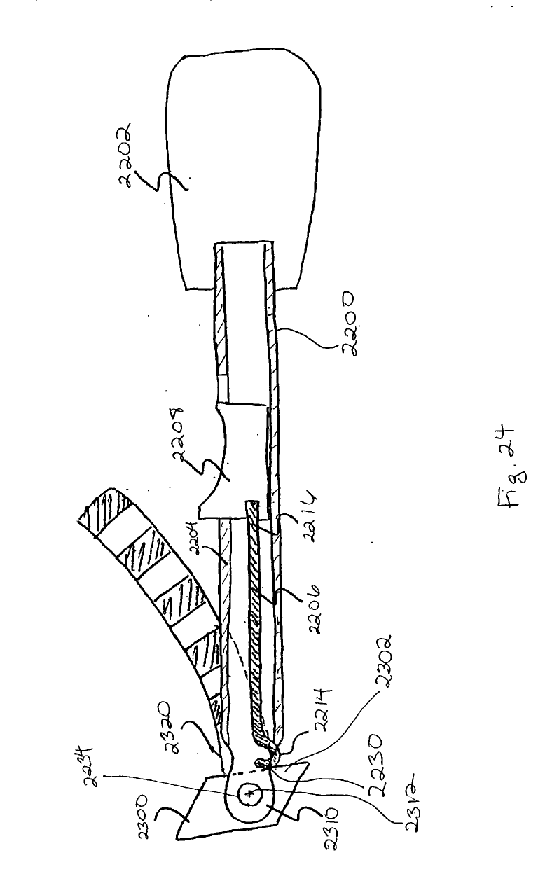

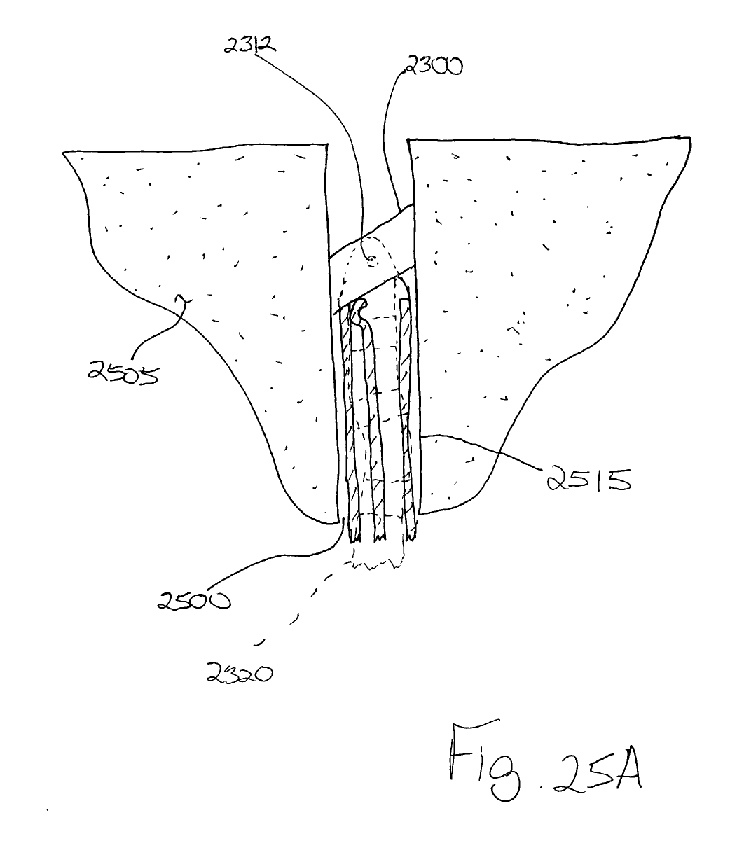

FIG. 20 illustrates a fixation mechanism 330 that includes spring loaded pins 332 and 334 disposed on a longitudinal shaft 336. FIG. 2P illustrates a fixation mechanism 340 that has a single spring-loaded pin 342 disposed on a longitudinal shaft 344.

FIG. 2Q illustrates a side view of a fixation mechanism 350 that includes rotary flyouts 352 extending from a longitudinal shaft 354. FIG. 2R illustrates a top view of the fixation mechanism 350 of FIG. 2Q.

FIG. 2S illustrates a fixation mechanism 360 with a pop rivet 362 disposed on a longitudinal shaft 364.

FIG. 2T illustrates a one-piece butterfly fixation mechanism 370 having a longitudinal shaft 372 disposed in the insertion tunnel and two fixation arms 374 and 376 that extend over the respective edges of the insertion tunnel after deployment of the fixation mechanism 370.

FIG. 2U illustrates a threaded push rod fixation mechanism 380 that is similar in shape and form to the push rod fixation mechanism 300 shown in FIG. 2K. However, the threaded push rod fixation mechanism 380 includes thread portions 388 and 389 on the respective interior, contacting portions of outer longitudinal shafts 382 and 386 and on the exterior of the interior longitudinal shaft 384. The longitudinal shafts 382 and 386 may be connected together at a proximal end (as shown in FIGS. 2E and 7).

FIG. 2V illustrates a threaded pull rod fixation mechanism 390 that is similar in shape and form to the pull rod fixation mechanism 230 shown in FIG. 2D. However, the respective interior contact points of longitudinal shafts 392 and 396 and interior longitudinal shaft 394 have threaded portions 398 and 399. The longitudinal shafts 392 and 396 may be connected together at a proximal end (as shown in FIGS. 2E and 7).

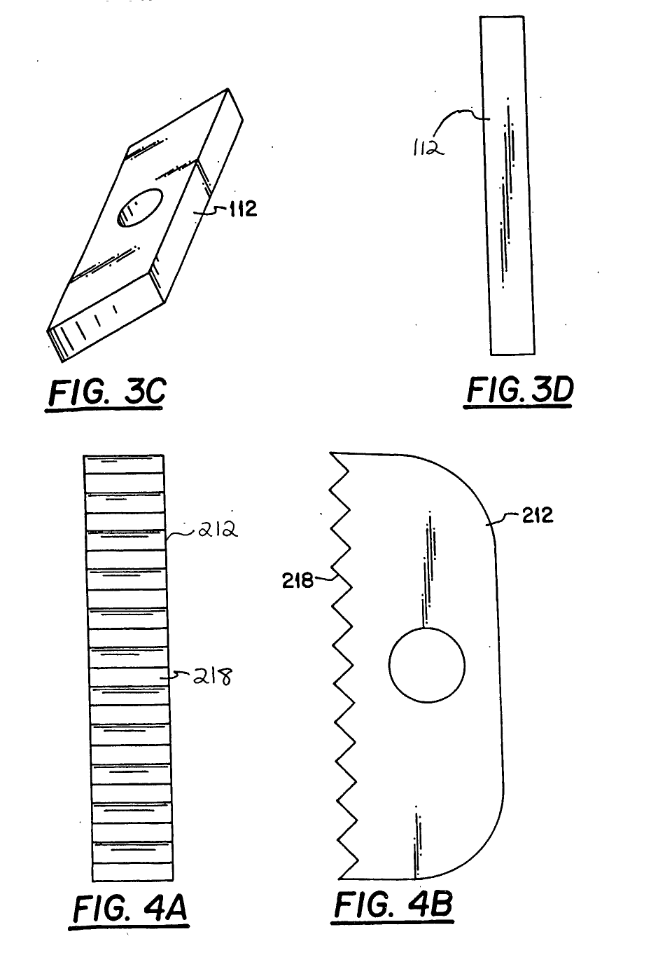

FIGS. 3A-3D show, respectively, front, top, perspective, and side views of the parallelogram-shaped fixation member 112 of the simple toggle fixation mechanism 110 shown in FIG. 2A.

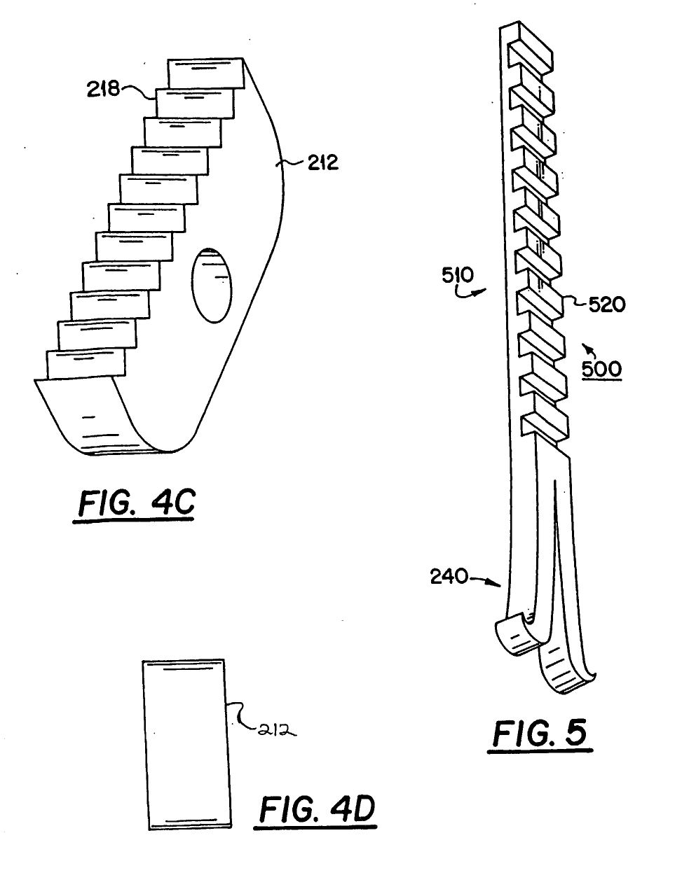

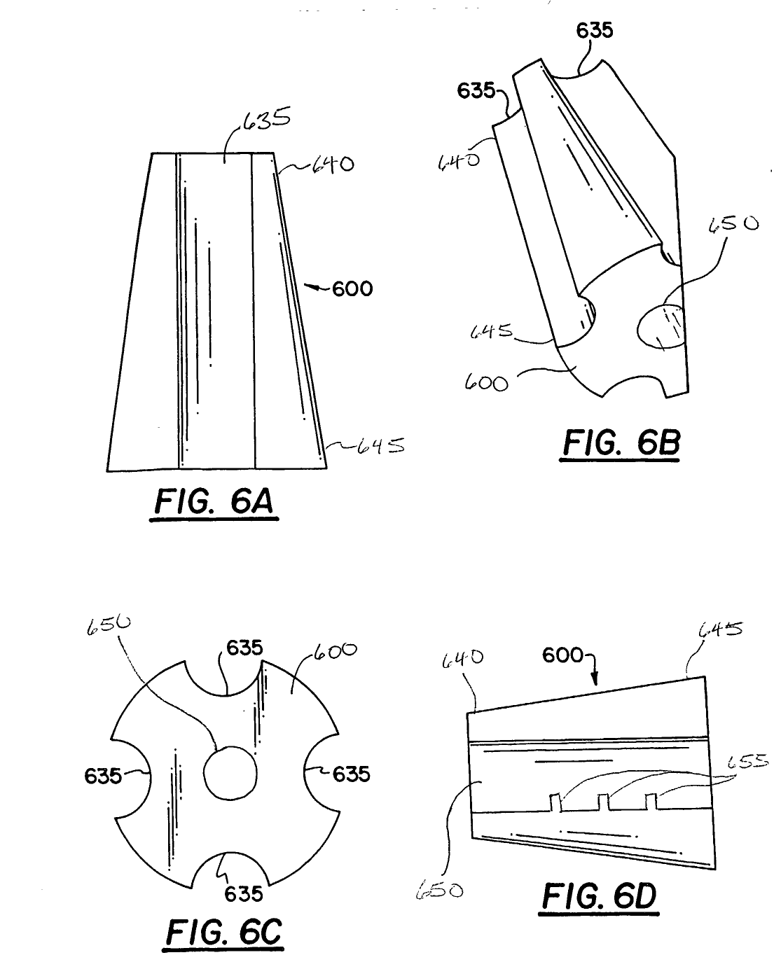

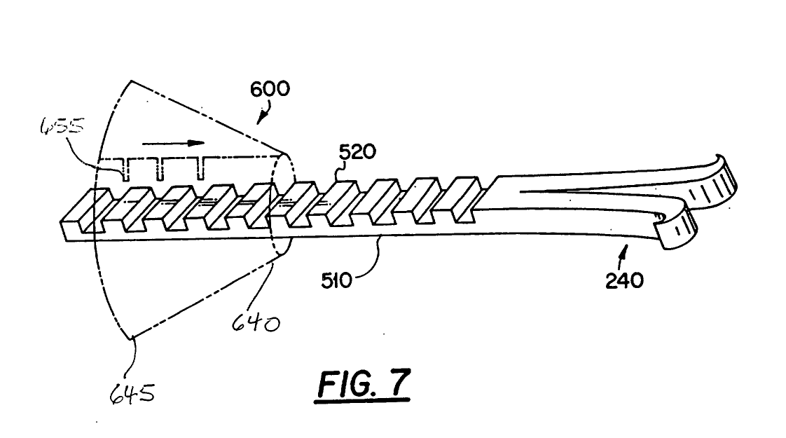

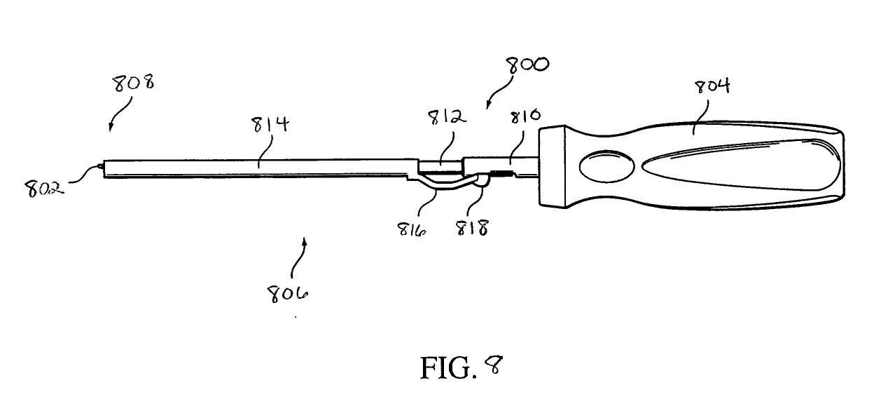

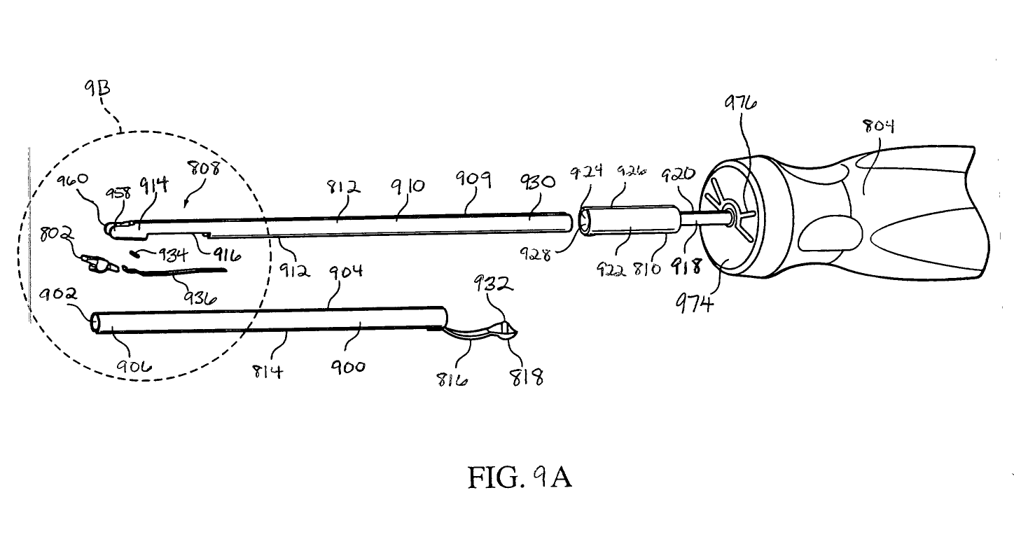

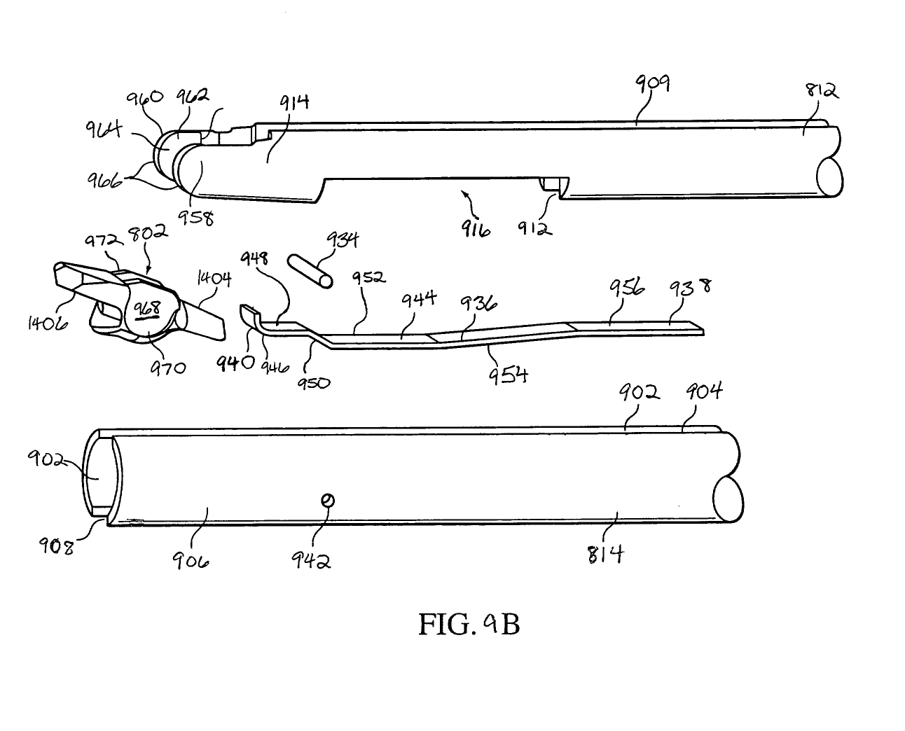

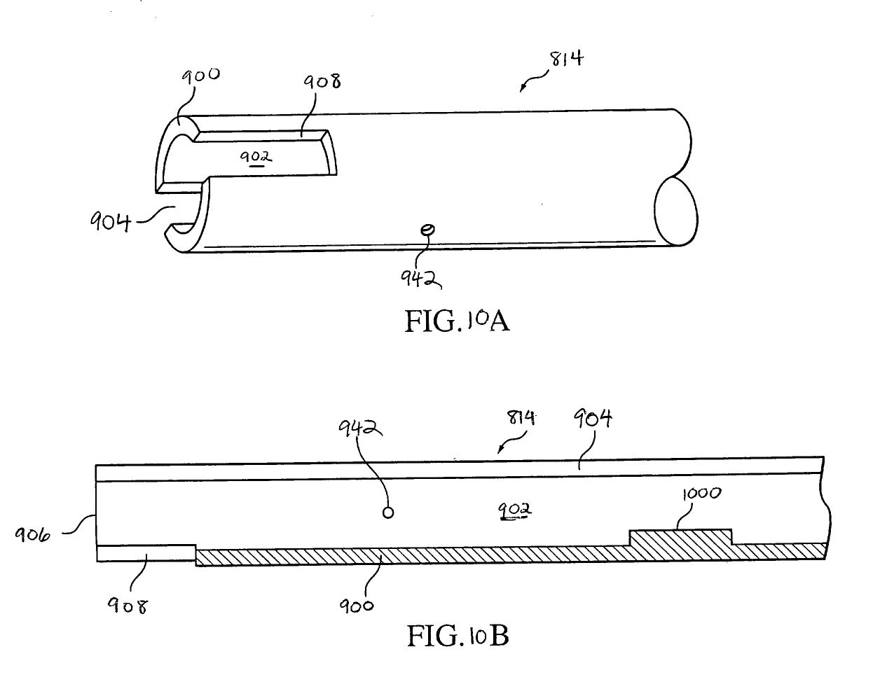

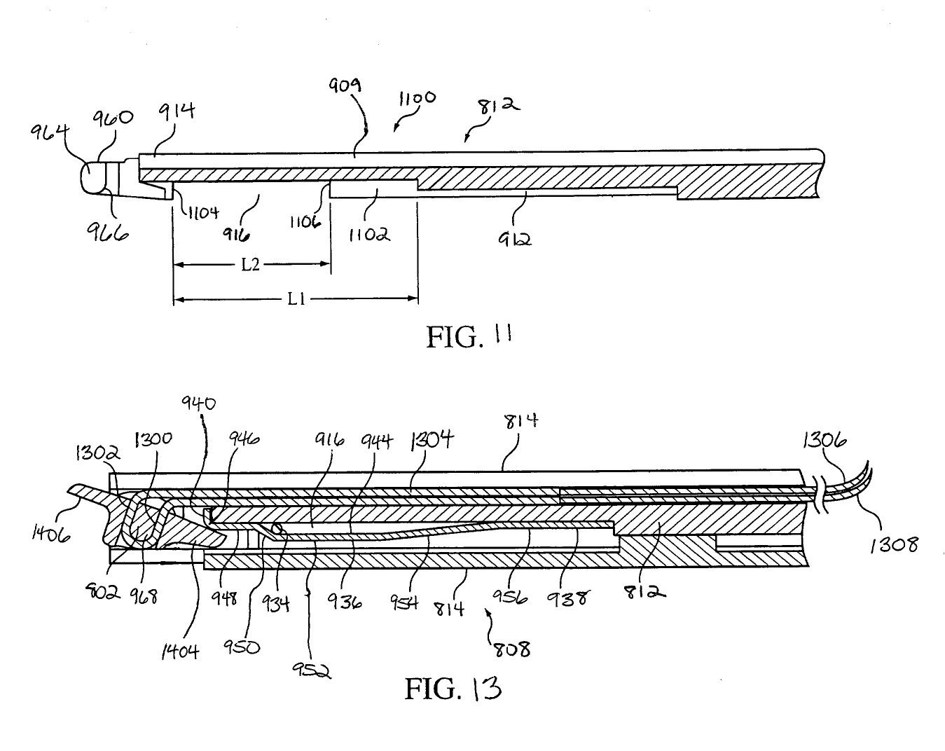

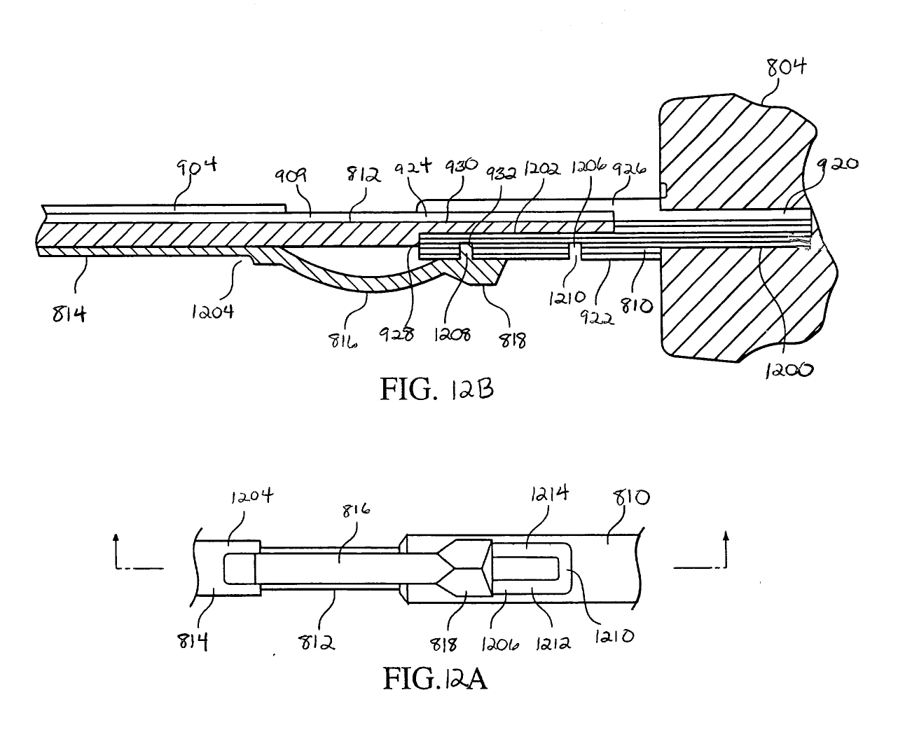

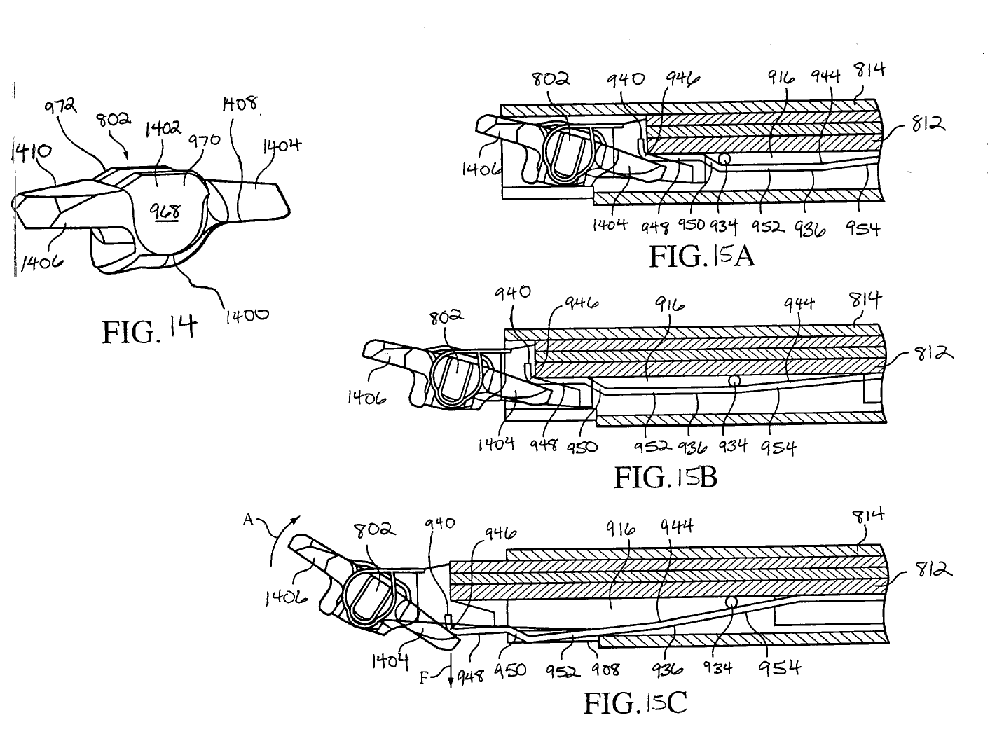

FIGS. 4A-4D show, respectively, side, front, perspective, and top views of the fixation member 212 of the alternative simple toggle fixation mechanism 210 shown in FIG. 2B.

FIG. 5 depicts the fixation mechanism 240 shown in FIG. 2E disposed on a shaft 510. The shaft 510 has raised portions 520 disposed thereon. Referring back to FIG. 1, the shaft 510 is disposed within the insertion tunnel such that the fixation mechanism 240 (in FIG. 1, fixation mechanism 110) protrudes from an end of the insertion tunnel. The raised portions 520 on the shaft 510 serve to provide a one-way track that prevents a closely-fit external sliding member, such as a securing mechanism, from reversing direction once the sliding member has started sliding on the shaft 5 1 0. The protrusions 520 may be replaced with, for example, pins, transverse tracks, bumps, or other elements for providing a one-way track.

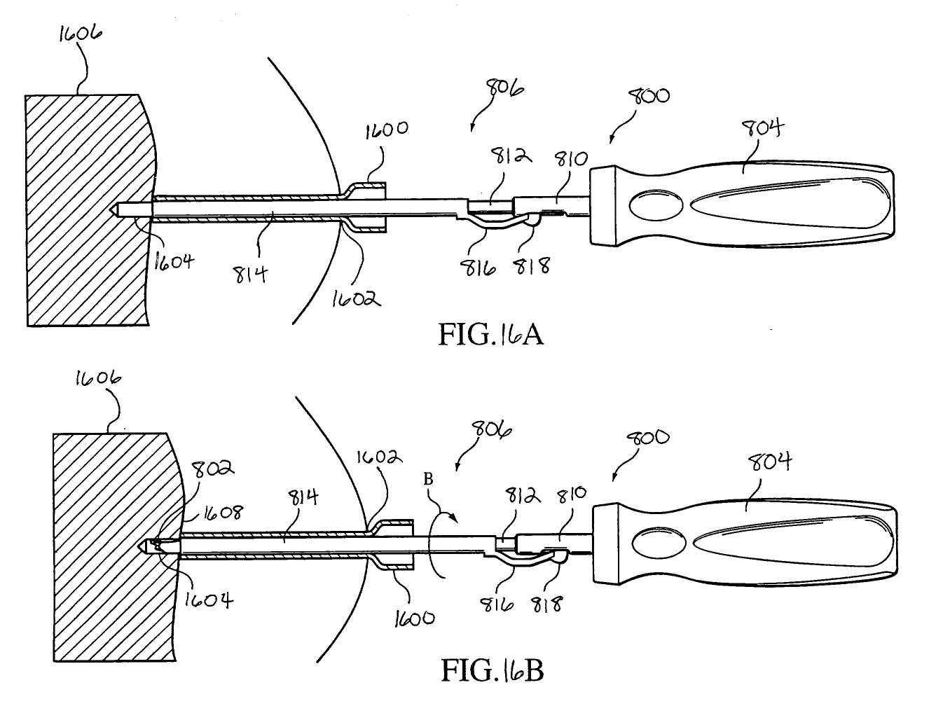

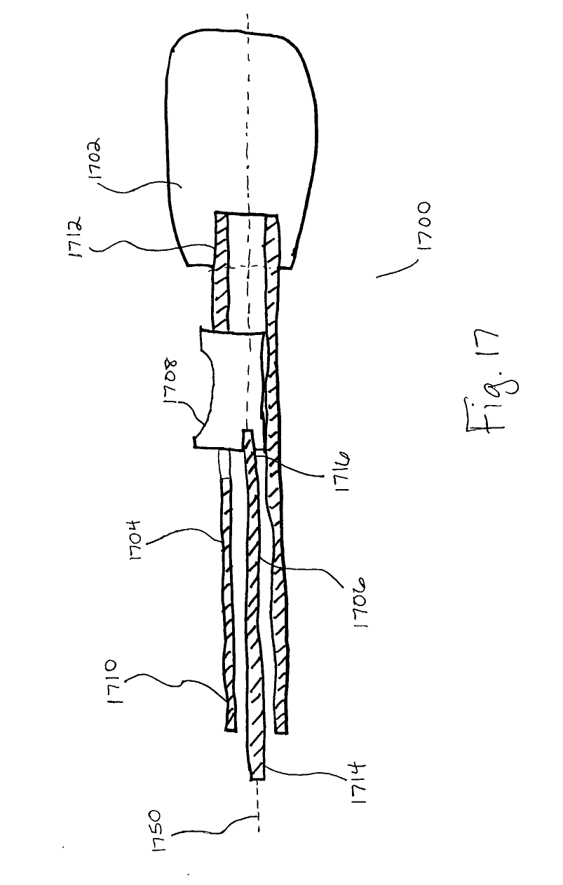

FIGS. 6A-6D show various views of a securing mechanism 600. Generally, a securing mechanism 600 has the shape of a plug with cutouts 635, designed to support tendon grafts without damage, about the circumference of the plug. FIG. 6A is a longitudinal side view of the securing mechanism 600. FIG. 6B is a schematic elevated perspective view of the securing mechanism 600 showing the cutouts 635 in the outer circumference of the securing mechanism 600. FIG. 6C is a schematic end view of the securing mechanism 600, showing the cutouts 635. FIG. 6D is a cross-sectional side view of the securing mechanism 600. The securing mechanism may be shaped as a cylinder, a cone, a cube, or some other complex shape that provides adequate radial force against the graft into the surrounding bone. As shown, the securing mechanism 600 may be formed with a tapered shape in which a tapered end 640 of the mechanism 600 is inserted into the insertion tunnel and a non-tapered end 645 lodges against the edge of the insertion tunnel to ensure that the securing mechanism 600 remains partially external to the insertion tunnel. The securing mechanism 600 includes a channel 650 for receiving the shaft 510. The channel 650 may include one or more raised portions 655 for mating with the raised portions 520 disposed on the shaft 510, as discussed below. The securing mechanism 600 may be made of any biocompatible metal, polymer, bioabsorbable polymer, or bone. If bone is used, an additional securing mechanism is used to provide the one-way locking on the shaft. The securing mechanism can transport site-specific drugs, such as bone morphing proteins, antibiotics, anti-inflammatories, and anesthetics.

FIG. 7 schematically depicts a fixation mechanism 240 on the shaft 510 and the securing mechanism 600 disposed together. The securing mechanism 600 is moved in the direction of the arrow along the shaft 510. The securing mechanism 600 engages with the shaft 510 to lock in place.

The surgeon loads the fixation device with the graft at full-length extension. The surgeon deploys fixation mechanism 240 within the insertion tunnel by first pushing the fixation mechanism into the insertion tunnel until it exits the insertion tunnel. The surgeon then pulls the fixation device proximally to prevent further retrograde movement.

After the surgeon deploys the fixation mechanism 240 within the insertion tunnel, the surgeon slides the tapered end 640 of the securing mechanism 600 over the shaft 510 such that the shaft 510 is inserted into the channel 650. The raised portions 655 of the channel 650 interfit with the raised portions 520 of the shaft 510 to effectively prevent the securing mechanism 600 from moving after being deployed on the shaft 510. The surgeon positions the graft 100 within the cutouts 635 of the securing mechanism 600 so that the graft 100 is between the wall of the insertion tunnel and the securing mechanism 600.

The surgeon then slides the securing mechanism 600 into the insertion tunnel until the non-tapered end 645 lodges within the insertion tunnel. When the non-tapered end 645 is lodged within the insertion tunnel, the graft 100 is pressed into the wall of the insertion tunnel to facilitate healing over a larger area. The securing mechanism 600 at the location of the cutouts 635 provides radial force to press the graft 100 against the wall of the insertion tunnel for faster and more efficient healing. Thus, the graft is held in place within the insertion tunnel by compression of the securing mechanism 600 against the graft 100 and is prevented from being pulled out of the insertion tunnel by the fixation mechanism 240. Once the femoral side of the graft 100 is pressed in place and locked on the shaft 510, the tibial end of the graft 100 is pulled into position and also locked into place.

The system of instrumentation and devices for attaching soft tissue to bone reduces the operating room time required to perform a procedure. The fixation device can be used in confined spaces, such as those in and around the human knee. The fixation device for attaching soft tissue to bone can be made of a bioabsorbable material, a biopolymer, or a biometal and can be easily removed and replaced. The devices can be deployed with one hand and allow the surgeon to individually tension each leg of an ACL graft. Using the system and/or device does not damage ACL grafts and a variety of graft sizes can be accommodated. Both ends of an ACL graft can be inserted through a single incision. Once the graft is in place, the securing mechanism radially presses the graft against the side of the insertion tunnel to provide increased graft-to-bone area and improve healing. Once deployed, the fixation device is secure within the tissue, and the force required to remove the fixation device is greater than the force (typically 800 Newtons) required to pull out standard interference screws. The system of instrumentation and devices for surgically securing an allograft or prosthetic ligament in a patient's bone are used in a procedure to replace a patient's cruciate ligament. As part of the replacement procedure for the anterior cruciate ligament, the patient's leg is bent at an approximately ninety (90°) degree angle and a single incision is made medial to the tibial tuberosity. Through this incision, an insertion tunnel is created at the desired insertion point of the graft using standard orthopedic techniques. The insertion tunnel exits on the lateral aspect of the femoral cortex. A replacement ligament is prepared using a hamstring allograft.

An insertion instrument allows the surgeon to deploy the fixation device with one hand. In particular, the surgeon is able to position the fixation mechanism, push the securing mechanism, and cut off the excess length of the graft using one hand.

In another implementation, instead of being disposed on a shaft 510, the fixation mechanism may be disposed on a suture and serve as a suture anchor.

Referring to FIG. 8, an anchor insertion tool 800 arthroscopically deploys a tissue anchor 802, for example, the RotorloC™ Anchor available from Smith & Nephew Endoscopy, Andover, Mass., by axially (longitudinally) advancing the anchor into a bone hole and applying a lateral force to the anchor to rotate the anchor. The tool 800 includes a handle 804 joined to an elongate portion 806 terminating in a distal region 808 that houses the tissue anchor 802. The elongate portion 806 includes an adapter 810 that is coupled to the handle 804, a shaft 812 coupled to the adapter 810, and a tubular cover 814 surrounding the shaft 812. The cover 814 is coupled to the adapter 810 to slide relative to the adapter 810, as described below. The tissue anchor 802 is located within the shaft 812 and is substantially covered by the cover 814 during introduction to a surgical site.

Referring also to FIGS. 9A, 9B, 10A and 10B, the cover 814 is a tubular member having a wall 900 defining a lumen 902 for receiving the shaft 812, and a slot 904 extending through the wall 900 along the entire length of the cover 814. Opposite to the slot 904, an additional slot 908 extends through the wall 900 over a length of about 5 to 15 mm (10 mm in one particular implementation) from a distal end 906 of the cover 814, for purposes described below. A guide 1000 extends from the wall 900 into the lumen 902.

Referring also to FIG. 11, the shaft 812 is a solid member with a first slot 909 and an opposite slot 912 in an exterior surface 910 of the shaft 812. The slot 909 extends the entire length of the shaft 812. The guide 1000 is received within the slot 912 to limit relative rotation between the shaft 812 and the cover 814 while allowing relative axial or longitudinal motion. The slot 912 extends up to about 150 mm (95 mm in one particular implementation) from a distal end 914 of the shaft 812, and the guide 1000 is spaced about 100 mm (65 mm in one particular implementation) from the distal end 906. The relative length of the slot 912 and the positioning of the guide 1000 provide clearance for a desired amount of relative axial or longitudinal motion between the shaft 812 and the cover 814.

The depth of the shaft slot 912 is increased in a distal region 1100 of the shaft 812 over a length L1 of about 20 to 50 mm (35 mm in one particular implementation) to form a chamber 1102 for purposes described below. The width of the shaft slot 912 is increased in the distal region 1100 of the shaft over a length L2 of about 10 to 30 mm (20 mm in one particular implementation) to form a cutout 916 having distal and proximal ends 1104, 1106, respectively, for purposes described below.

Referring to FIGS. 9A and 12B, the adapter 810 includes a coupling portion 918 received within a bore 1200 in the handle 804 and fixed to the handle 804 by, for example, epoxy. The coupling portion 918 defines a slot 920. The adapter 810 has a wall 922 defining a bore 924 and a slot 926 extending from the bore 924 through the wall 922. The slot 926 is aligned with the slot 920. Opposite to the slot 926, an axial nub 928 extends from the wall 922 into the bore 924 and runs the length of the adapter 810. The shaft 812 has an additional slot 1202 opposite the slot 909. The slot 1202 receives the nub 928 when a proximal end 930 of the shaft 812 is slid into the bore 924. The placement of the nub 928 within the slot 1202 limits relative rotation between the shaft 812 and the adapter 810.

Referring to FIGS. 12A and 12B, the cover 814 is coupled to adaptor 810 by a resilient thumb contact 816. The contact 816 extends from a proximal end 1204 of the cover 814 to a guide channel 1206 defined in the adapter 810. The contact 816 includes a mating member 818 supporting a nub 932 that is received in the guide channel 1206. The guide channel 1206 has a race-track shape with proximal and distal portions 1208, 1210, respectively, and side portions 1212, 1214. In an unstressed state, the contact 816 is straight with the nub 932 in the middle of the portion 1208 or 1210. To axially move the cover 814, the operator flexes the contact 816 sideways to align the nub 932 with the side portion 1212 or 1214 and moves the nub 932 axially along the side portion 1212 or 1214. When the nub 932 has been moved the full length of the side portion, the contact 816 springs back to a straight orientation returning the nub 932 to the middle of the portion 1208 or 1210. This spring action provides positive control on the relative motion between the cover 814 and the shaft 812. The distance between the proximal and distal portions 1208, 1210 is, for example, about 10 to 20 mm (15 mm in one particular implementation) and defines the range over which the cover 814 can be slid relative to the shaft 812.

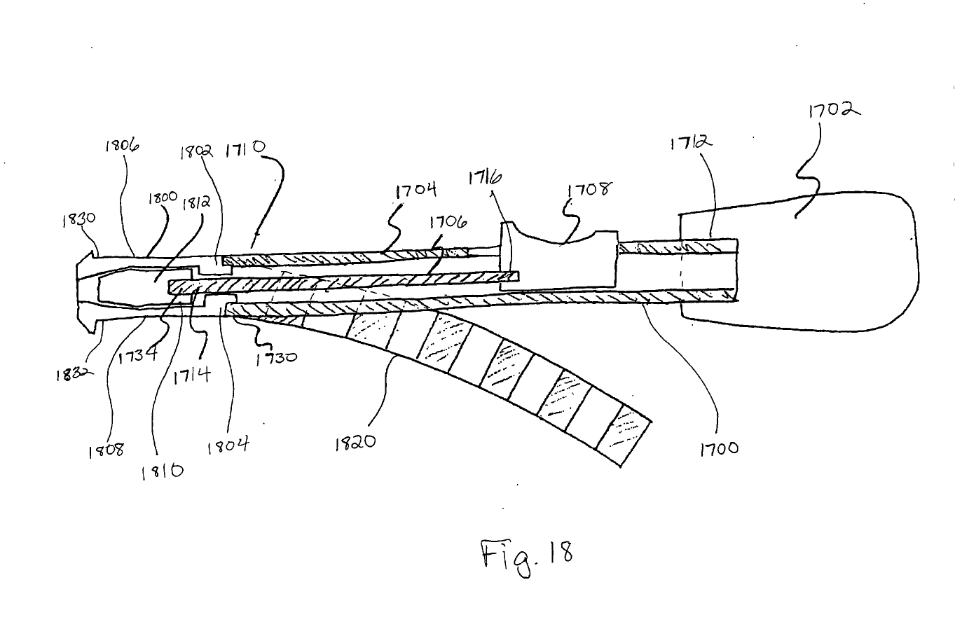

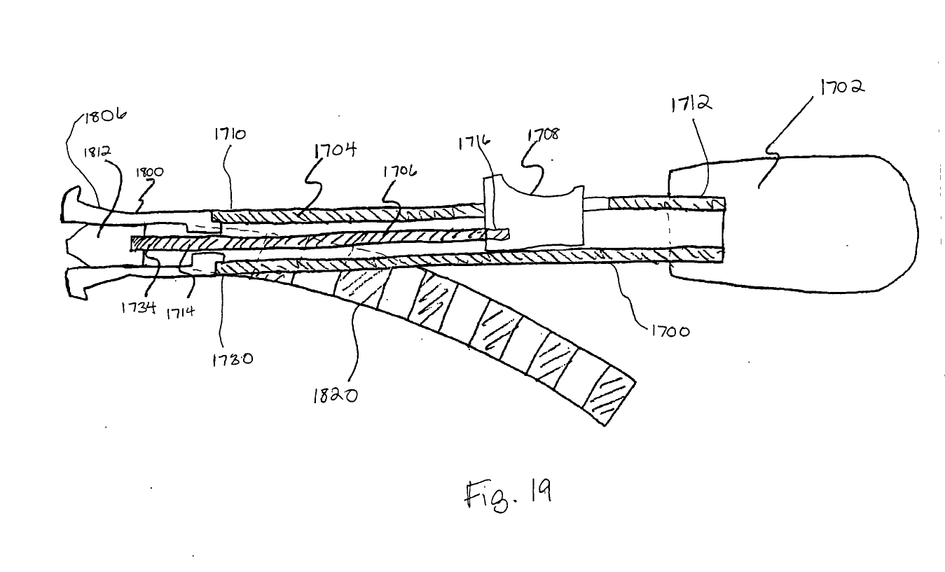

Referring to FIGS. 9A, 9B, and 13, the cover 814 includes a flexor in the form of a pin 934 and the shaft 812 includes an applicator in the form of a spring 936 located within the chamber 1102. The pin 934 and the spring 936 couple the cover 814 and the shaft 812 such that retraction of the cover 814 relative to the shaft 812 causes lateral deflection of the spring 936, as described below. The spring 936 is received within the shaft chamber 1102 and has a proximal end 938 attached to the shaft 812 by for example, epoxy, and a free distal end 940. The cover 814 defines a pair of opposing holes 942 in which the pin 934 is received such that the pin 934 extends through the lumen 902. As shown in FIG. 13, the pin 934 is received within the cutout 916 between the shaft 812 and the spring 936 and contacts a surface 944 of the spring 936. The length of the cutout 916 provides clearance for desired axial motion of the pin 934.

The spring 936 is contoured to control lateral flexing of the spring 936 as the pin 934 is moved along the surface 944 of the spring 936. From the distal end 940 to the proximal end 938, the spring 936 includes an arcuate portion 946 that engages the anchor 802, a straight portion 948, a sloped portion 950, a straight portion 952, a sloped portion 954, and a straight portion 956. When the cover 814 is moved relative to the shaft 812, the pin 934 slides along the surface 944 of the spring 936. When the pin 934 engages the portion 954 of the spring 936, the spring 936 deflects laterally, which moves the distal end 940 of the spring 936 laterally against the anchor 802 to deploy the anchor 802 from the tool 800, as described further below.

The shaft 812 includes a pair of opposing, spaced apart arms 958 and 960 that define an anchor receiving region 962 therebetween. Each of the arms 958 and 960 has an internal pivot face 964 bounded by an arcuate edge 966. The tissue anchor 802 is coupled to the shaft 812 by placement between arms 958 and 960 in abutment with the faces 964. The free end 938 of the spring 936 extends into the region 962 and contacts the anchor 802.

Referring also to FIG. 14, the tissue anchor 802 includes a central portion 968 with an opposing pair of pivoting faces 970 and 972. Each of the pivoting faces 970 and 972 includes a raised arcuate lip 1400 with a radius of curvature substantially equal to the radius of curvature of arcuate edges 966 of arms 958 and 960. When assembled, the faces 964 of the arms 958 and 960 are positioned against the anchor faces 970 and 972, with edges 966 against lips 1400. Due to the shapes of the edges 966 and the lips 1400, the anchor 802 can rotate relative to arms 958 and 960. The lip 1400 does not define a complete circle about faces 970 and 972 such that the anchor 802 has an opening 1402 to each of the faces 970 and 972. When the anchor 802 is slid between the arms 958 and 960, the arm pivot faces 964 pass through the openings 1402 into position against the anchor faces 970 and 972. The anchor 802 is maintained in position between the arms 958 and 960 by the engagement of the lips 1400 with the edges 966, and by the positioning of the cover 814 about the anchor 802.

The tissue anchor 802 includes a pair of wings 1404 and 1406 with, respectively, oppositely oriented, angled cutting edges 1408 and 1410. As shown in FIG. 13, the central portion 968 of the tissue anchor 802 defines a pair of suture channels 1300 and 1302 for receiving two suture strands 1304 (with only one suture strand being shown). When assembled, with the suture strands 1304 threaded through the channels 1300 and 1302, each suture strand 1304 passes between the arms 958 and 960 to the slot 909, and along the slot 909 to the adapter slot 920. At the end of each suture strand there is a needle 1306 and 1308. The handle 804 has a face 974 defining four slots 976 (FIG. 9A) in which the needles are located during introduction of the anchor 802 into tissue.

Referring to FIGS. 15A and 16A, during introduction of the tool 800 into tissue, the mating member 818 is in contact with the distal face 1210 of the guide channel 1206 and the pin 934 is near the distal end 1104 of the cutout 916 such that the cover 814 is disposed distally to substantially cover the tissue anchor 802. The spring 936 rests against tissue anchor 802 without exerting a lateral force on the anchor, and the pin 934 contacts the face 944 of the spring 936 at the junction of the spring portions 950 and 952. The position of the cover 814 over the anchor 802 limits possible dislodgement of the anchor 802 from the tool 800 during introduction into the tissue, and protects the tissue from the anchor.

Referring to FIG. 15B, to deploy the anchor, the operator first slides the member 818 and thus the cover 814 proximally to a position near the middle of the slidable range (that is, the member 818 is near the middle of the guide channel 1206 and the pin 934 is near the middle of the cutout 916). The pin 934 now contacts the spring 936 at the junction of the spring portions 952 and 954, and the anchor 802 is partially uncovered. Since the spring portion 952 is oriented parallel to the axis of the elongate portion 806, the movement of the pin 934 does not deflect the spring 936 and the spring 936 still rests against the tissue anchor 802 without exerting a lateral force on the anchor.

Referring to FIG. 15C and 16B, to rotate the anchor 802 (arrow A), the operator slides the member 818 and thus the cover 814 further proximally such that the member 818 is in contact with the proximal face 1208 of the guide channel 1206 and the pin 934 is near the distal end 1106 of the cutout 916. The anchor 802 is now fully uncovered. The movement of the pin 934 along the sloped spring portion 954 laterally deflects the spring 936. The spring portions 950 and 952 are received within the cover slot 908, and the distal spring portion 946 exerts a substantially laterally directed force, F, on the anchor 802 to cause the anchor to rotate. The rotation of the tissue anchor 802 pivots the anchor 802 within the arms 958 and 960. The proximal translation of the cover 814 thus both exposes and rotates the anchor 802.

Referring to FIGS. 16A and 16B, in use, for example, in shoulder repair, with a cannula 1600 placed through a skin portal 1602, the operator advances the tissue anchor insertion tool 800 through the cannula 1600 to a predrilled hole 1604 in a tissue 1606, such as, for example, bone tissue. The operator then moves the member 818 proximally to the channel portion 1210, which move the cover 814 proximally, while pushing the insertion tool 800 into the hole 1606. This results in the shaft 812 entering the bone hole with the distal end of the cover 814 abutting a bone surface 1608, and the anchor 802 is uncovered and rotated, as described above, with the ends of the tissue anchor wings 1404 and 1406 starting to push into the bone tissue surrounding the hole 1604. The operator then applies a torque to the handle 804 to rotate the insertion tool 800 and the tissue anchor 802, in the direction of arrow B. The applied torque causes the edges 1408 and 1410 of the anchor 802 to cut into the bone tissue, and, because the cutting edges are set at an angle, the rotation of the anchor 802 along arrow B results in additional rotation of the anchor 802 along arrow A (as shown in FIG. 15C). About 1½ turns of the tool 800 rotates the anchor 802 such that the anchor wings 1404 and 1406 are embedded in the bone tissue and oriented substantially perpendicular to the bone wall. The rotation of the anchor 802 to this perpendicular position aligns the anchor face openings 1402 with the arms 958 and 960 such that the arms 958 and 960 can be slid from the anchor 802 through the openings 1402. Thus, to release the anchor 802 from the shaft 812, the operator simply moves the tool 800 proximally.

Referring to FIG. 17, an insertion instrument 1700 is used with the fixation mechanisms 230, 300, 380, and 390 shown, respectively, in FIGS. 2D, 2K, 2S, and 2T. For simplicity, the following discussion refers to use of the insertion instrument with a fixation mechanism and a disposed shaft. It should be recognized that the insertion instrument may be used with a suture anchor and a disposed suture.

The insertion instrument 1700 includes a handle 1702, a main member 1704 extending along a longitudinal axis 1750, a secondary member 1706, and a pushing handle 1708. The main member 1704 has a distal end 17 10 and a proximal end 1712. The distal end 1710 is formed to accept and hold the fixation mechanism. The proximal end 1712 is configured to be firmly held within the instrument handle 1702. The secondary member 1706 has a distal end 1714 and a proximal end 1716. The main member 1704 also includes a channel through which a shaft disposed on the fixation mechanism passes during deployment of the fixation mechanism.

Referring also to FIG. 18, a fixation mechanism 1800 is mounted to the insertion instrument 1700 before the surgeon deploys the fixation mechanism 1800 into the insertion tunnel. The distal end 1710 of the main member 1704 includes a feature 1730 that mates with proximal ends 1802 and 1804 of outer longitudinal members 1806 and 1808 respectively, of the fixation mechanism 1800. The distal end 1714 of the secondary member 1704 includes a feature 1734 that interacts with a proximal end 18 10 of an inner longitudinal member 1812 of the fixation mechanism 1800. The fixation mechanism 1800 is disposed on the proximal ends 1802 and 1804 of the shaft 1820 and the shaft 1820 exits the channel of the main member 1704. The outer longitudinal members 1806 and 1808 include distal ends 1830 and 1832 that are able to expand relative to proximal ends 1802 and 1804, respectively.

Referring also to FIGS. 19, 20, and 21A-21C, the surgeon performs a procedure 2000 using the insertion instrument 1700. Typically, the fixation mechanism 1800 is attached to the insertion instrument 1700 during manufacturing and thus the surgeon need not attach the mechanism to the instrument. However, in the rare case that manufacturing does not include this attachment, the surgeon attaches the fixation mechanism 1800 to the insertion instrument 1700 (step 2005). In either case, attachment includes fixing the proximal ends 1802 and 1804 to the feature 1730 and fixing the proximal end 1810 to the feature 1734 by, for example, snap fit, mating threads, glue, or interference fit. At this or a later time, the surgeon may attach the graft 100 to the fixation mechanism using the securing mechanism (step 2007).

The surgeon creates the insertion tunnel 2100 within tissue 2105 of a patient (step 2010) by, for example, drilling a hole into the patient's tissue 2105. The surgeon then positions the insertion instrument 1700 with the fixation mechanism 1800 in the insertion tunnel 2100 (step 2015). Next, the surgeon guides the fixation mechanism 1800 through the insertion tunnel 2100 (FIG. 21A) until the fixation mechanism 1800 clears the insertion tunnel 2100 (step 2020). Thus, the surgeon translates the pushing handle 1708 along the longitudinal axis relative to the instrument handle 1702. When the surgeon translates the pushing handle 1708 along the longitudinal axis 1750 relative to the instrument handle 1702, the translation causes the secondary member 1706 to longitudinally move relative to the main member 1704, thus causing the inner longitudinal member 1812 to move relative to the outer longitudinal members 1806 and 1808 (FIGS. 19 and 21B).

The surgeon translates the pushing handle 1708 by, for example, gripping the instrument handle 1702 with her fingers and placing her thumb on the pushing handle 1708. The movement of the inner longitudinal member 1812 relative to the outer longitudinal members 1806 and 1808 expands the outer longitudinal members 1806 and 1808 beyond the radius of the insertion tunnel 2100 to prevent further movement of the fixation mechanism 1800 relative to the tissue 2105.

Thus, if the surgeon translates the pushing handle 1708 after positioning the distal ends 1830 and 1832 of the outer longitudinal members 1806 and 1808 beyond the edge of the insertion tunnel 2100, the outer longitudinal members 1806 and 1808 are expanded such that the distal ends 1830 and 1832 engage an outer wall 2110 of the tissue 2105 that is not facing the insertion tunnel 2100 (FIG. 21B). If the surgeon translates the pushing handle 1708 while the distal ends 1830 and 1832 remain in the tissue 2105, the distal ends 1830 and 1832 expand into a wall 2115 of the insertion tunnel 2100 to fix the fixation mechanism 1800 within the tissue 2105.

At this or a later time, the surgeon may use the insertion instrument or any suitable device to apply a force and a counterforce to adjust or apply tension to the shaft so that the securing mechanism moves along the shaft and into the insertion tunnel 2100 to secure the graft 100 (step 2022).

Next, the surgeon releases the insertion instrument 1700 from the fixation mechanism 1800 (step 2025) by, for example, pulling the insertion instrument 1700 away from the fixation mechanism 1800 (FIG. 21C). Because of this releasing motion, the surgeon pulls back on the outer longitudinal members 1806 and 1808, which then engage the outer wall 2110. This results in a smooth, simple release of the suture, fixation mechanism, pre-attached needles, and the shaft from the insertion instrument 2200. The surgeon then continues the repair. For example, the surgeon may remove excess portions of the shaft 1820 as necessary (step 2030).

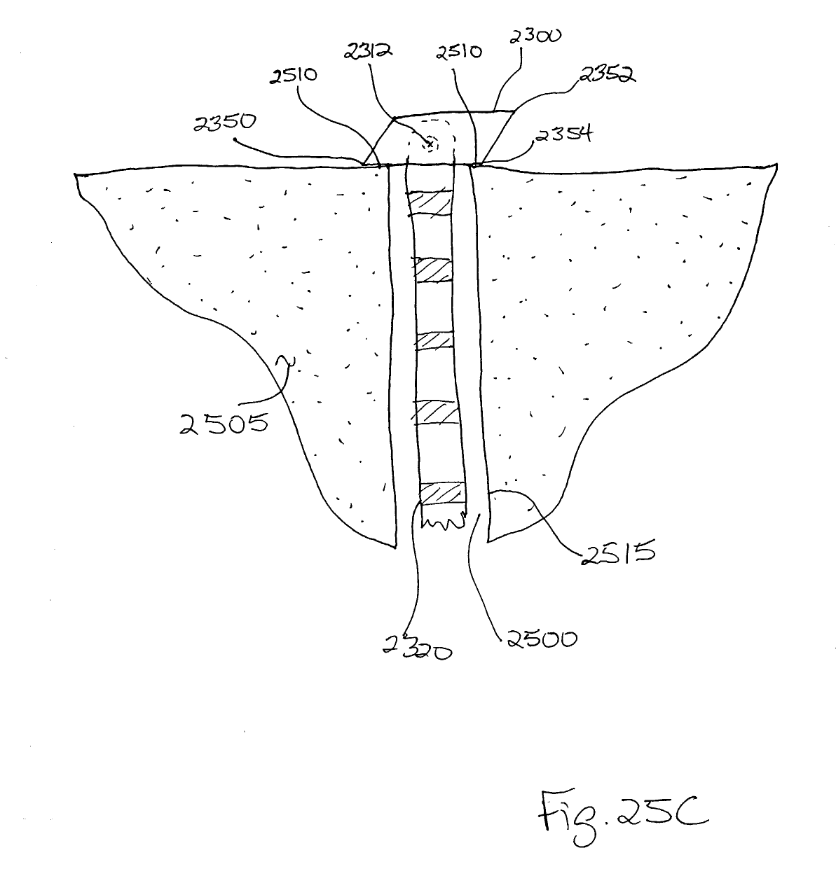

Referring to FIG. 22, an insertion instrument 2200 is used with the fixation mechanisms shown in FIGS. 2A-2C, 2F, 2G, and 2R. For simplicity, the following discussion refers to use of the insertion instrument with a fixation mechanism and a disposed shaft. It should be recognized that the insertion instrument may be used with a suture anchor and a disposed suture.

The insertion instrument 2200 includes a handle 2202, a main member 2204 extending along a longitudinal axis 2250, a secondary member 2206, and a pushing handle 2208. The main member 2204 has a distal end 2210 and a proximal end 2212. The distal end 2210 is formed to accept and hold the fixation mechanism. The proximal end 2212 is configured to be firmly held within the instrument handle 2202. The secondary member 2206 has a distal end 2214 and a proximal end 2216. The main member 2204 also includes a channel through which a shaft disposed on the fixation mechanism passes during deployment of the fixation mechanism.

Referring also to FIG. 23, a fixation mechanism 2300 is mounted to the insertion instrument 2200 before the surgeon deploys the fixation mechanism 2300 into the insertion tunnel. The distal end 2210 of the main member 2204 includes a feature 2234 that mates with first portion 2310 of the fixation mechanism 2300. The distal end 2214 of the secondary member 2206 includes a feature 2230 that interacts with a second portion 2302 of the fixation mechanism 2300. The fixation mechanism 2300 is disposed on the shaft 2320 and the shaft 2320 exits a side channel (not shown though discussed above) of the main member 2204. The second portion 2302 is able to rotate relative to the first portion 2310 because the fixation mechanism 2300 is secured to the feature 2234 at a pivot point 2312 and because the second portion 2302 is free to move.

Referring again to FIG. 20 and also to FIGS. 24 and 25A-25C, the surgeon performs the procedure 2000 for using the insertion instrument 2200. As discussed, the fixation mechanism 2300 is typically attached to the insertion instrument 2200 during manufacture and thus the surgeon need not perform the attachment at step 2005. However, in the rare case that manufacturing does not include this attachment, the surgeon attaches the fixation mechanism 2300 to the insertion instrument 2200 (step 2005). In either case, attachment includes fixing the first portion 2310 to the feature 2234 by, for example, snap fit, mating threads, glue, or interference fit.

The surgeon creates an insertion tunnel 2500 within tissue 2505 of a patient (step 2010) by, for example, drilling a hole into the patient's tissue 2505. The surgeon then positions the insertion instrument 2200 with the fixation mechanism 2300 into the insertion tunnel 2500 (step 2015). Next, the surgeon guides the fixation mechanism 2300 through the insertion tunnel 2500 (FIG. 25A) until the fixation mechanism 2300 clears the insertion tunnel 2500 (step 2020). For example, the surgeon translates the pushing handle 2208 along the longitudinal axis 2250 relative to the instrument handle 2202. When the surgeon translates the pushing handle 2208 along the longitudinal axis 2250 relative to the instrument handle 2202, the translation causes the second member 1706 to longitudinally move relative to the main member 1704, thus causing the second portion 2302 to rotate (as depicted by arrow 2400 in FIG. 23) relative to the first portion 2310 at the pivot point 2312 (FIGS. 23 and 25B).

The surgeon translates the pushing handle 2208 by, for example, gripping the instrument handle 2202 with her fingers and placing her thumb on the pushing handle 2208. The rotation of the second portion 2302 relative to the first portion 2310 about the pivot point 2312 causes acute corners 2350, 2352 to extend beyond the radius of the insertion tunnel 2500 to prevent further movement of the fixation mechanism 2300 relative to the tissue 2505.

Thus, if the surgeon translates the pushing handle 2208 after positioning the fixation mechanism 2300 beyond the edge of the insertion tunnel 2500, the acute corner 2350 and an obtuse corner 2354 engage an outer wall 2510 of the tissue 2505 that is not facing the insertion tunnel 2500 (FIG. 25C). If the surgeon translates the pushing handle 2208 while the fixation mechanism 2300 remains in the tissue 2505, the acute corners 2350, 2352 are expanded into a wall 2515 of the insertion tunnel 2500 to fix the fixation mechanism 2300 within the tissue 2505.

After deploying the fixation mechanism 2300 as desired, the surgeon simply releases the insertion instrument 2200 (step 2025) by, for example, pulling the insertion instrument 2200 longitudinally away from the fixation mechanism 2300 (FIG. 25C). Because of this releasing motion, the surgeon pulls back on the fixation mechanism 2300, which then engages the outer wall 2510. This results in a smooth, simple release of the suture, fixation mechanism, pre-attached needles, and the shaft from the insertion instrument 2200. The surgeon then continues the repair.

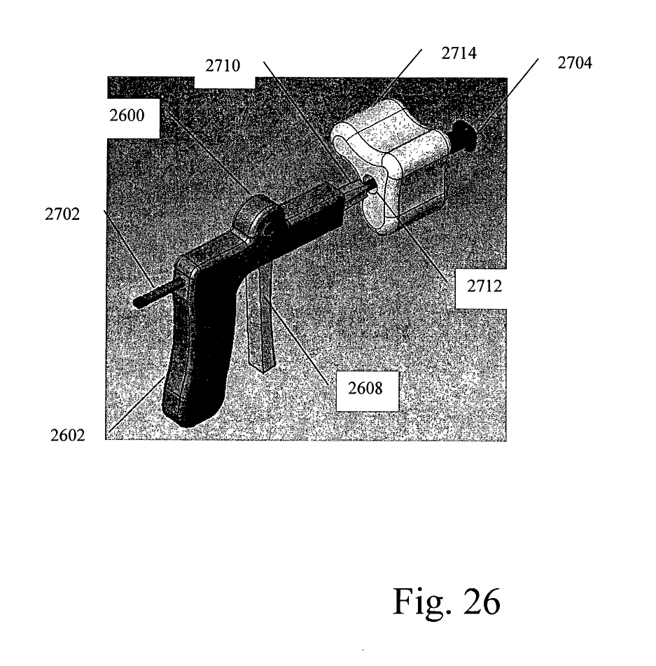

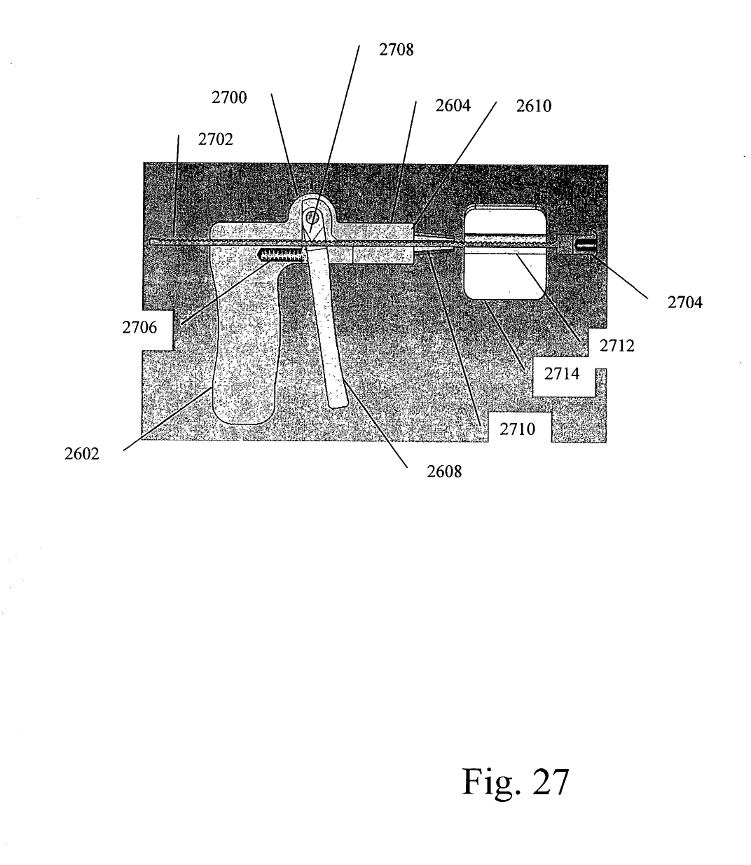

Referring to FIGS. 26-28, an insertion instrument 2600 may be used with any of the fixation mechanisms described in FIGS. 2A-2V. For simplicity, the following discussion refers to use of the insertion instrument 2600 with a pull rod such as the pull rod of FIG. 2D. It should be recognized that the insertion instrument 2600 also may be used with a suture anchor and a disposed suture.

The insertion instrument 2600 includes a handle 2602, a main member 2604 extending perpendicularly from the handle 2602 and along a longitudinal axis 2650, and a handle 2608 that moves relative to the handle 2602 and the main member 2604. The main member 2604 includes a channel 2700 extending along the axis 2650 and having a size large enough to accommodate the shaft 2702 attached to a fixation mechanism 2704. The main member 2604 also includes a biasing device 2706 for biasing the handle 2608 away from the handle 2602. The handle 2608 includes a tensioning device 2708 for engaging the raised portions of the shaft 2702 to enable the surgeon to control the tension of the fixation mechanism 2704 relative to a securing mechanism 2710 on the shaft 2702, as discussed below. The tensioning device 2708 may be a ratchet device such as a tooth that engages the raised portions of the shaft 2702. The main member 2604 also includes a distal surface 2610.

Referring again to FIG. 20, the surgeon performs the procedure 2000 for using the insertion instrument 2600. Typically, the fixation mechanism 2704 is attached to the insertion instrument 2600 during manufacturing such that the surgeon does not need to attach the mechanism to the instrument. However, in the rare case that manufacturing does not include this attachment, the surgeon attaches the fixation mechanism 2704 to the insertion instrument 2600 by inserting the shaft 2702 (to which the fixation mechanism 2704 is attached) through the channel 2700 when the tooth 2708 of the handle 2608 is disengaged from the shaft 2702 (step 2005). At this or a later time, the surgeon may attach the graft 100 using the securing mechanism 2710 (step 2007).

The surgeon creates an insertion tunnel 2712 within tissue 2714 of a patient (step 2010) by, for example, drilling a hole into the patient's tissue 2714. The surgeon then positions the insertion instrument 2600 with the fixation mechanism 2704 in the insertion tunnel 2712 (step 2015). Next, the surgeon guides the fixation mechanism 2704 through the insertion tunnel 2712 until the fixation mechanism 2704 clears or is positioned in the appropriate location within the insertion tunnel 2712 (as detailed above) (step 2020).

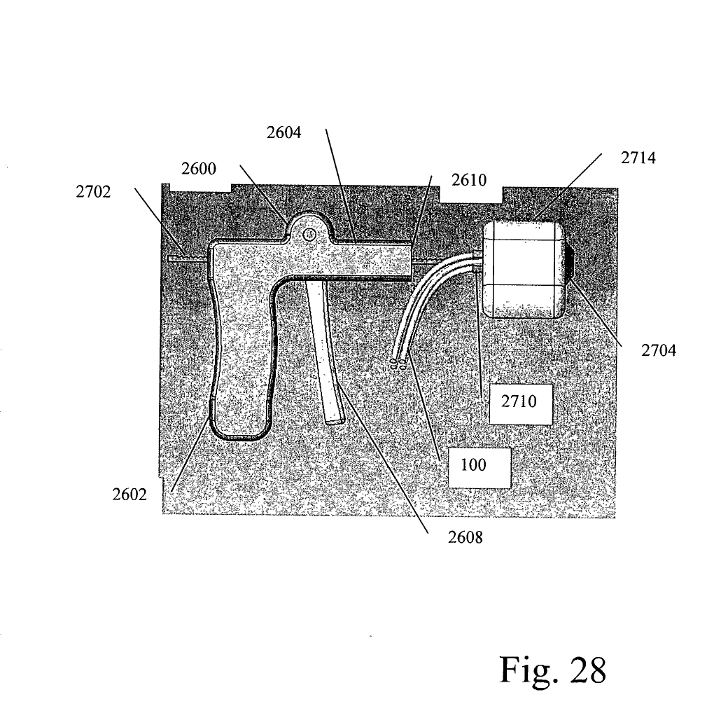

After the fixation mechanism 2704 is appropriately positioned relative to the insertion tunnel 2712, the surgeon uses the insertion instrument 2600 to apply a force and a counterforce to adjust or apply tension to the shaft 2702 so that the securing mechanism 2710 moves along the shaft 2702 and into the insertion tunnel 2712 to secure the graft 100 (step 2022). For example, the surgeon may periodically push the handle 2608 relative to the handle 2602 to ratchet the shaft 2702 proximally. This ratcheting causes the securing mechanism 2710 (on which the graft 100 is deployed) to enter the insertion tunnel 2712 because the shaft 2702 moves proximally while the securing mechanism 2710 is prevented from moving in a distal direction along the longitudinal axis when the securing mechanism 2710 contacts the distal surface 2610. Eventually, after the surgeon applies enough force, the graft 100, which has been positioned at the securing mechanism 2710, is squeezed into the wall of the insertion tunnel 2712 by the securing mechanism 2710, which becomes lodged between the distal surface 2610 and within the insertion tunnel 2712. The ratcheting of the shaft 2702 also causes the fixation mechanism 2704, which is secured to the shaft 2702, to move proximally to grip or wedge into the tissue 2714 (as shown in FIG. 28).

Next, the surgeon releases the insertion instrument 2600 from the fixation mechanism 2704 and the shaft 2702 (step 2025) by, for example, disengaging the tooth 2708 and pulling the insertion instrument 2600 away from the fixation mechanism 2704. This results in a smooth, simple release of the suture, the fixation mechanism, the pre-attached needles, and the shaft from the insertion instrument 2600. The surgeon then continues the repair. For example, the surgeon may remove excess shaft 2702 as necessary (step 2030).

Manufacture of the insertion instrument may be done using a number of processes including machining and molding from biocompatible metal(s) and polymer(s).

Other implementations are within the scope of the following claims.

What is claimed is:

1. A method of deploying a fixation mechanism, the method comprising:
providing an insertion instrument having a main member connected to an instrument handle and a secondary member connected to a pushing handle and positioned within the main member, the main member and the secondary member extending along a longitudinal axis;
providing a fixation mechanism having a first portion coupled to the main member and a second portion coupled to the secondary member;
creating an insertion tunnel within tissue of a patient;
positioning the insertion instrument with the fixation mechanism in the insertion tunnel; and
translating the pushing handle along the longitudinal axis relative to the instrument handle such that translating causes the secondary member to move the second portion relative to the first portion.

2. The method of claim 1 in which translating causes the secondary member to translate the second portion along the longitudinal axis relative to the first portion.

3. The method of claim 2 in which translating causes distal ends of the first portion to expand along a direction perpendicular to the longitudinal direction to a size having a radius greater than a radius of the insertion tunnel.

4. The method of claim 3 in which translating causes distal ends of the first portion to enter tissue adjacent the insertion tunnel.

5. The method of claim 3 in which translating causes distal ends of the first portion to engage tissue external the insertion tunnel.

6. The method of claim 1 in which translating causes the secondary member to rotate the second portion relative to the first portion about an axis perpendicular to the longitudinal axis.

7. The method of claim 6 in which translating causes a width of the second portion to expand after rotation relative to a width before rotation to a width greater than a radius of the insertion tunnel.

8. The method of claim 7 in which translating causes ends of the second portion to enter tissue adjacent the insertion tunnel.

9. The method of claim 7 in which translating causes ends of the second portion to engage tissue external the insertion tunnel.

10. A system for deploying a fixation mechanism, the system comprising:
an insertion instrument having a main member connected to an instrument handle and a secondary member connected to a pushing handle and positioned within the main member, the main member and the secondary member extending along a longitudinal axis; and
a fixation mechanism having a first portion coupled to the main member and surrounding a second portion coupled to the secondary member;
wherein the insertion instrument and the fixation mechanism are coupled such that translation of the pushing handle along the longitudinal axis relative to the instrument handle causes the secondary member to move the second portion relative to the first portion and to expand distal ends of the first portion along a direction perpendicular to the longitudinal axis.

11. A method for deploying a graft, the method comprising:
providing an insertion instrument having a main member connected to a main handle and a tensioning device connected to a tensioning handle and positioned within the main member, the main member extending along a longitudinal axis;
providing a securing mechanism coupled to a shaft of a fixation mechanism;
coupling the shaft to the tensioning device;
positioning the fixation mechanism in an insertion tunnel of tissue;
using the tensioning device, providing tension between the fixation mechanism and the securing mechanism to deploy a graft within the insertion tunnel.

12. A system for deploying a fixation mechanism, the system comprising:
an insertion instrument having a main member connected to a main handle and a tensioning device positioned within the main member and coupled to a tensioning handle that is able to move relative to the main handle, the main member extending along a longitudinal axis;
a shaft connected to the fixation mechanism and coupled to the tensioning device of the main member; and
a securing mechanism coupled to the shaft at a distal end of the insertion instrument, wherein the shaft is coupled to the tensioning device and connected to the fixation mechanism such that translation of the tensioning handle relative to the main handle causes the securing mechanism to move distally relative to the shaft and to deploy a graft coupled to the securing mechanism.

13. The system of claim 12 in which the tensioning handle and the main handle extend in a perpendicular direction from the longitudinal axis.

14. The system of claim 12 in which the tensioning device includes a ratchet for coupling to raised portions of the shaft.

15. The system of claim 12 in which the main member defines a channel for receiving the shaft and the tensioning device.

16. The system of claim 12 in which the main member includes a biasing device that biases the tensioning handle at a position away from the main handle.
